SEQUENCE LISTING (70 sequences)

SEQ ID NO: 1 - protein, 9 aa, oryctolagus cuniculus
LSLSSNTLN

SEQ ID NO: 2 - protein, 9 aa, oryctolagus cuniculus
LSLSTNAPN

SEQ ID NO: 3 - protein, 10 aa, oryctolagus cuniculus
FSLSNNYWIC

SEQ ID NO: 4 - protein, 10 aa, oryctolagus cuniculus
FSFSSGYDMC

SEQ ID NO: 5 - protein, 10 aa, oryctolagus cuniculus
FSLSDSAYMC

SEQ ID NO: 6 - protein, 10 aa, oryctolagus cuniculus
FNVNSDCYMC

SEQ ID NO: 7 - protein, 10 aa, oryctolagus cuniculus
FSFFYDYYMC

SEQ ID NO: 8 - protein, 19 aa, oryctolagus cuniculus
WIGTIFIDDEDTYYASWAK

SEQ ID NO: 9 - protein, 19 aa, oryctolagus cuniculus
WIGTIFIDDEDTYYASWAK

SEQ ID NO: 10 - protein, 19 aa, oryctolagus cuniculus
WIACIYTGRDYTYYTSWAK

SEQ ID NO: 11 - protein, 20 aa, oryctolagus cuniculus
WIACIYPGSSGNTYYASWAK

SEQ ID NO: 12 - protein, 20 aa, oryctolagus cuniculus
WIACIDAGVSGSTYYASWVN

SEQ ID NO: 13 - protein, 19 aa, oryctolagus cuniculus
WIGCIDTVSGSTYYATWAK

SEQ ID NO: 14 - protein, 19 aa, oryctolagus cuniculus
WIGCIYGASGDTYYASWAK

SEQ ID NO: 15 - protein, 11 aa, oryctolagus cuniculus
YFCARSSDLWD

SEQ ID NO: 16 - protein, 11 aa, oryctolagus cuniculus
YFCARNSDLWD

SEQ ID NO: 17 - protein, 15 aa, oryctolagus cuniculus
YFCARNNTDYGDYWD

SEQ ID NO: 18 - protein, 20 aa, oryctolagus cuniculus
YFCARTYASSSGDYIPYFFN

SEQ ID NO: 19 - protein, 19 aa, oryctolagus cuniculus
YFCARDFLYTPNNAGDIMD

SEQ ID NO: 20 - protein, 19 aa, oryctolagus cuniculus
YFCARDFLYNVNNAGDIMD

SEQ ID NO: 21 - protein, 16 aa, oryctolagus cuniculus
YFCARDVDTGSGIDFE

SEQ ID NO: 22 - protein, 11 aa, oryctolagus cuniculus
QSVYKNNYLSW

SEQ ID NO: 23 - protein, 11 aa, oryctolagus cuniculus
QSVYNNNYLSW

SEQ ID NO: 24 - protein, 11 aa, oryctolagus cuniculus
QSVYKNNYLAW

SEQ ID NO: 25 - protein, 9 aa, oryctolagus cuniculus
ENIYSGLAW

SEQ ID NO: 26 - protein, 11 aa, oryctolagus cuniculus
HTVYSNNYLSW

SEQ ID NO: 27 - protein, 11 aa, oryctolagus cuniculus
YNVYNNNYLAW

SEQ ID NO: 28 - protein, 9 aa, oryctolagus cuniculus
QSIGSYLSW

SEQ ID NO: 29 - protein, 12 aa, oryctolagus cuniculus
LIYQASKLASGV

SEQ ID NO: 30 - protein, 12 aa, oryctolagus cuniculus
LIYKASTLASGV

SEQ ID NO: 31 - protein, 12 aa, oryctolagus cuniculus
LIYWASKLPSGV

SEQ ID NO: 32 - protein, 12 aa, oryctolagus cuniculus
LIYDASDLASGV

SEQ ID NO: 33 - protein, 12 aa, oryctolagus cuniculus
LIYSASSLASGV

SEQ ID NO: 34 - protein, 12 aa, oryctolagus cuniculus
LIYTTSTLASGV

SEQ ID NO: 35 - protein, 12 aa, oryctolagus cuniculus
LIYSTSTLASGV

SEQ ID NO: 36 - protein, 15 aa, oryctolagus cuniculus
LGSYDCRSANCIVFG

SEQ ID NO: 37 - protein, 15 aa, oryctolagus cuniculus
LGSYDCRSANCIVFG

SEQ ID NO: 38 - protein, 15 aa, oryctolagus cuniculus
LGSYDCTIAECNVFG

SEQ ID NO: 39 - protein, 14 aa, oryctolagus cuniculus
QNYYYSSRGFNTFG

SEQ ID NO: 40 - protein, 12 aa, oryctolagus cuniculus
HGYWRGPVNDFG

SEQ ID NO: 41 - protein, 12 aa, oryctolagus cuniculus
QGYYRGPVNDFG

SEQ ID NO: 42 - protein, 14 aa, oryctolagus cuniculus
QQGDTNHNIDNIFG

SEQ ID NO: 43 - protein, 112 aa, oryctolagus cuniculus
QQQLEESGGGLVKPGGTLTLTCTVSGLSLSSNTLNWVRQAPGKGLEWIGTIFIDDEDTYYASWAKGRFTISKTSSTTITLKMTSLTAADTATYFCARSSDLWDPGTLVVVSS

SEQ ID NO: 44 - protein, 111 aa, oryctolagus cuniculus
QSLEESGGGLVKPGGTLTLTCTVSGLSLSTNAPNWVRQAPGKGLEWIGTIFIDDEDTYYASWAKGRFTISKTSSTTITLKMTSLTAADTATYFCARNSDLWDPGTLVVVSS

SEQ ID NO: 45 - protein, 120 aa, oryctolagus cuniculus
QEHLEESGGGLVKPGASLTLTCTASGFSLSNNYWICWVRQAPGKGLEWIACIYTGRDYTYYTSWAKGRFTISKTSSTTVTLQLNSLTAADTATYFCARNNTDYGDYWDLWGPGTLVTVSS

SEQ ID NO: 46 - protein, 126 aa, oryctolagus cuniculus
QQQVVESGGGLVKPGASLTLTCEASGFSFSSGYDMCWVRQAPGKGLEWIACIYPGSSGNTYYASWAKGRFTISKTSSTTVTLQMTSLTAADTATYFCARTYASSSGDYIPYFFNLWGPGTLVTVSS

SEQ ID NO: 47 - protein, 125 aa, oryctolagus cuniculus
QEQLEESGGGLVQPEGSLTLTCTASGFSLSDSAYMCWVRQAPGKGLEWIACIDAGVSGSTYYASWVNGRFTISKTSSTTVTLQMTSLTVADTATYFCARDFLYTPNNAGDIMDLWGPGTLVTVSL

SEQ ID NO: 48 - protein, 124 aa, oryctolagus cuniculus
QQQLEESGGGLVQPGGSLTLSCKASGFNVNSDCYMCWVRQAPGKGLEWIGCIDTVSGSTYYATWAKGRFTISKTSSTTVTLQMTSLTAADTATYFCARDFLYNVNNAGDIMDLWGPGTLVTVSL

SEQ ID NO: 49 - protein, 120 aa, oryctolagus cuniculus
QSLEESGGDLVQPEGTLTLTCTASGFSFFYDYYMCWVRQAPGKGLEWIGCIYGASGDTYYASWAKGRFTISKTSSTTVTLRMTSLTAADTATYFCARDVDTGSGIDFELWGPGTLVTVSS

SEQ ID NO: 50 - protein, 113 aa, oryctolagus cuniculus
AQVLTQTPSSVSAAVGGTVTINCQASQSVYKNNYLSWFQQKPGQPPKLLIYQASKLASGVSSRFKGSGSGTQFTLTISDVQCDDAATYFCLGSYDCRSANCIVFGGGTEVVVK

SEQ ID NO: 51 - protein, 113 aa, oryctolagus cuniculus
AQVLTQTPSPVSAAVGGTVTINCQASQSVYNNNYLSWFQQKPGQPPKLLIYKASTLASGVSSRFKGSGSGTQFTLTISDVQCDDAATYFCLGSYDCRSANCIVFGGGTEVVVK

SEQ ID NO: 52 - protein, 113 aa, oryctolagus cuniculus
AQVLTQTASPVSVAVGDTVTINCQASQSVYKNNYLAWYQLKPGQPPKLLIYWGTKLPSGVPSRFKGSGSGTHFTLTISDVQCDDAATYYCLGSYDCTIAECNVFGGGTEVVVE

SEQ ID NO: 53 - protein, 111 aa, oryctolagus cuniculus
ADIVMTQTPASVEAAVGGTVTIKCQASENIYSGLAWYQQKPGQPPKLLIYDASDLASGVPSRFKGSGSGTEYTLTISDLECADAATYYCQNYYYSSRGFNTFGGGTEVVVK

SEQ ID NO: 54 - protein, 110 aa, oryctolagus cuniculus
AQVLTQTPSSVSAAVGGTVTINCQSSHTVYSNNYLSWYQQKPGQPPKLLIYSASSLASGVPSRFKGSGSGTQFTLTISDLECDDAAIYYCHGYWRGPVNDFGGGTEVVVE

SEQ ID NO: 55 - protein, 110 aa, oryctolagus cuniculus
AQVLTQTPSSVSAAVGGTVTINCQSSYNVYNNNYLAWYQQKPGQPPKLLIYTTSTLASGVPSRFSGSGSGTQFTLTISDLECDDAAIYYCQGYYRGPVNDFGGGTEVVVE

SEQ ID NO: 56 - protein, 110 aa, oryctolagus cuniculus
AYDMTQTPASVEVAVGGTVTIKCQASQSIGSYLSWYQQKPGQPPKLLIYSTSTLASGVPSRFKGSGSGTQFTLTISGVECADAATYYCQQGDTNHNIDNIFGGGTEVVVE

SEQ ID NO: 57 - protein, 207 aa, oryctolagus cuniculus
QQQLEESGGGLVKPGGTLTLTCTVSGLSLSSNTLNWVRQAPGKGLEWIGTIFIDDEDTYYASWAKGRFTISKTSSTTITLKMTSLTAADTATYFCARSSDLWDPGTLVVVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 58 - protein, 206 aa, oryctolagus cuniculus
QSLEESGGGLVKPGGTLTLTCTVSGLSLSTNAPNWVRQAPGKGLEWIGTIFIDDEDTYYASWAKGRFTISKTSSTTITLKMTSLTAADTATYFCARNSDLWDPGTLVVVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 59 - protein, 215 aa, oryctolagus cuniculus
QEHLEESGGGLVKPGASLTLTCTASGFSLSNNYWICWVRQAPGKGLEWIACIYTGRDYTYYTSWAKGRFTISKTSSTTVTLQLNSLTAADTATYFCARNNTDYGDYWDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 60 - protein, 221 aa, oryctolagus cuniculus
QQQVVESGGGLVKPGASLTLTCEASGFSFSSGYDMCWVRQAPGKGLEWIACIYPGSSGNTYYASWAKGRFTISKTSSTTVTLQMTSLTAADTATYFCARTYASSSGDYIPYFFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 61 - protein, 220 aa, oryctolagus cuniculus
QEQLEESGGGLVQPEGSLTLTCTASGFSLSDSAYMCWVRQAPGKGLEWIACIDAGVSGSTYYASWVNGRFTISKTSSTTVTLQMTSLTVADTATYFCARDFLYTPNNAGDIMDLWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 62 - protein, 219 aa, oryctolagus cuniculus
QQQLEESGGGLVQPGGSLTLSCKASGFNVNSDCYMCWVRQAPGKGLEWIGCIDTVSGSTYYATWAKGRFTISKTSSTTVTLQMTSLTAADTATYFCARDFLYNVNNAGDIMDLWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 63 - protein, 215 aa, oryctolagus cuniculus
QSLEESGGDLVQPEGTLTLTCTASGFSFFYDYYMCWVRQAPGKGLEWIGCIYGASGDTYYASWAKGRFTISKTSSTTVTLRMTSLTAADTATYFCARDVDTGSGIDFELWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 64 - protein, 217 aa, oryctolagus cuniculus
AQVLTQTPSSVSAAVGGTVTINCQASQSVYKNNYLSWFQQKPGQPPKLLIYQASKLASGVSSRFKGSGSGTQFTLTISDVQCDDAATYFCLGSYDCRSANCIVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 65 - protein, 217 aa, oryctolagus cuniculus
AQVLTQTPSPVSAAVGGTVTINCQASQSVYNNNYLSWFQQKPGQPPKLLIYKASTLASGVSSRFKGSGSGTQFTLTISDVQCDDAATYFCLGSYDCRSANCIVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 66 - protein, 217 aa, oryctolagus cuniculus
AQVLTQTASPVSVAVGDTVTINCQASQSVYKNNYLAWYQLKPGQPPKLLIYWGTKLPSGVPSRFKGSGSGTHFTLTISDVQCDDAATYYCLGSYDCTIAECNVFGGGTEVVVEGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 67 - protein, 215 aa, oryctolagus cuniculus
ADIVMTQTPASVEAAVGGTVTIKCQASENIYSGLAWYQQKPGQPPKLLIYDASDLASGVPSRFKGSGSGTEYTLTISDLECADAATYYCQNYYYSSRGFNTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 68 - protein, 214 aa, oryctolagus cuniculus
AQVLTQTPSSVSAAVGGTVTINCQSSHTVYSNNYLSWYQQKPGQPPKLLIYSASSLASGVPSRFKGSGSGTQFTLTISDLECDDAAIYYCHGYWRGPVNDFGGGTEVVVEGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 69 - protein, 214 aa, oryctolagus cuniculus
AQVLTQTPSSVSAAVGGTVTINCQSSYNVYNNNYLAWYQQKPGQPPKLLIYTTSTLASGVPSRFSGSGSGTQFTLTISDLECDDAAIYYCQGYYRGPVNDFGGGTEVVVEGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 70 - protein, 214 aa, oryctolagus cuniculus
AYDMTQTPASVEVAVGGTVTIKCQASQSIGSYLSWYQQKPGQPPKLLIYSTSTLASGVPSRFKGSGSGTQFTLTISGVECADAATYYCQQGDTNHNIDNIFGGGTEVVVEGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

(19) United States
(12) Patent Application Publication
(10) Pub. No.: US 2022/0056112 A1
(43) Pub. Date: Feb. 24, 2022

(54) MONOCLONAL ANTIBODIES AGAINST SARS-CoV-2 NUCLEOCAPSID PROTEIN AND USES THEREOF

(71) Applicant: YUROGEN BIOSYSTEMS LLC, Worcester, MA (US)

(72) Inventors: Hai WU, Worcester, MA (US); Yang LOU, Worcester, MA (US)

(21) Appl. No.: 17/399,367

(22) Filed: Aug. 11, 2021

Related U.S. Application Data
(60) Provisional application No. 63/064,090, filed on Aug. 11, 2020.

(51) Int. Cl. C07K 16/10; G01N 33/543; C07K 19/00; C12N 9/08

(52) U.S. Cl. CPC C07K 16/10; G01N 33/543; C07K 19/00; C12Y 111/01007; C07K 2317/565; C07K 2317/24; G01N 2333/165; C12N 9/0065

(57) ABSTRACT

The disclosure provides rabbit monoclonal antibodies against the nucleocapsid protein of SARS-CoV-2 and uses thereof. The antibody comprises: a V_{H} CDR1 selected from the group consisting of SEQ ID NO: 1-7; a V_{H} CDR2 selected from the group consisting SEQ ID NO: 8-14; a V_{H} CDR3 selected from the group consisting of SEQ ID NO: 15-21; a V_{L} CDR1 selected from the group consisting SEQ ID NO: 22-28; a V_{L} CDR2 selected from the group consisting of SEQ ID NO: 29-35; and a V_{L} CDR3 selected from the group consisting of SEQ ID NO: 36-42. The antibodies can be used for a rapid test or screening of SARS-CoV-2 infection and detecting SARS-CoV-2 nucleocapsid protein.

### FIELD

The disclosure relates to the rabbit monoclonal antibodies (mAbs) against SARS-CoV-2. Specifically, the disclosure relates to a panel of rabbit monoclonal antibodies against the nucleocapsid protein (NP) of SARS-CoV-2 and use thereof.

### BACKGROUND

The SARS-CoV-2 refers to the severe acute respiratory syndrome coronavirus 2 (SARS-CoV-2) initially identified in December 2019 that causes coronavirus disease 2019 (COVID-19). The NP of SARS-CoV-2 is a structural protein that forms complexes with the genomic RNA, interacts with the viral membrane protein during virion assembly, and plays a critical role in enhancing virus transcription and assembly efficiency.

### SUMMARY

Embodiments provide monoclonal antibodies against the SARS-CoV-2 NP and use thereof. In an embodiment, the monoclonal antibody comprises a V_{H} CDR1 having the amino acid sequence of SEQ ID NO: 1, a V_{H} CDR2 having the amino acid sequence of SEQ ID NO: 8, a V_{H} CDR3 having the amino acid sequence of SEQ ID NO: 15, a V_{L} CDR1 having the amino acid sequence of SEQ ID NO: 22, a V_{L} CDR2 having the amino acid sequence of SEQ ID NO: 29, and a V_{L} CDR3 having the amino acid sequence of SEQ ID NO: 36. In an embodiment, the monoclonal antibody comprises a V_{H} CDR1 having the amino acid sequence of SEQ ID NO: 2, a V_{H} CDR2 having the amino acid sequence of SEQ ID NO: 9, V_{H} CDR3 having the amino acid sequence of SEQ ID NO: 16, a V_{L} CDR1 having the amino acid sequence of SEQ ID NO: 23, a V_{L} CDR2 having the amino acid sequence of SEQ ID NO: 30, and a V_{L} CDR3 having the amino acid sequence of SEQ ID NO: 37. In an embodiment, the monoclonal antibody comprises a V_{H} CDR1 having the amino acid sequence of SEQ ID NO: 3, a V_{H} CDR2 having the amino acid sequence of SEQ ID NO: 10, V_{H} CDR3 having the amino acid sequence of SEQ ID NO: 17, a V_{L} CDR1 having the amino acid sequence of SEQ ID NO: 24, a V_{L} CDR2 having the amino acid sequence of SEQ ID NO: 31, and a V_{L} CDR3 having the amino acid sequence of SEQ ID NO: 38. In an embodiment, the monoclonal antibody comprises a V_{H} CDR1 having the amino acid sequence of SEQ ID NO: 4, a V_{H} CDR2 having the amino acid sequence of SEQ ID NO: 11, a V_{H} CDR3 having the amino acid sequence of SEQ ID NO: 18, a V_{L} CDR1 having the amino acid sequence of SEQ ID NO: 25, a V_{L} CDR2 having the amino acid sequence of SEQ ID NO: 32, and a V_{L} CDR3 having the amino acid sequence of SEQ ID NO: 39. In an embodiment, the monoclonal antibody comprises a V_{H} CDR1 having the amino acid sequence of SEQ ID NO: 5, a V_{H} CDR2 having the amino acid sequence of SEQ ID NO: 12, a V_{H} CDR3 having the amino acid sequence of SEQ ID NO: 19, a V_{L} CDR1 having the amino acid sequence of SEQ ID NO: 26, a V_{L} CDR2 having the amino acid sequence of SEQ ID NO: 33, and a V_{L} CDR3 having the amino acid sequence of SEQ ID NO: 40. In an embodiment, the monoclonal antibody comprises a V_{H} CDR1 having the amino acid sequence of SEQ ID NO: 6, a V_{H} CDR2 having the amino acid sequence of SEQ ID NO: 13, a V_{H} CDR3 having the amino acid sequence of SEQ ID NO: 20, a V_{L} CDR1 having the amino acid sequence of SEQ ID NO: 27, a V_{L} CDR2 having the amino acid sequence of SEQ ID NO: 34, and a V_{L} CDR3 having the amino acid sequence of SEQ ID NO: 41. In an embodiment, the monoclonal antibody comprises a V_{H} CDR1 having the amino acid sequence of SEQ ID NO: 7, a V_{H} CDR2 having the amino acid sequence of SEQ ID NO: 14, a V_{H} CDR3 having the amino acid sequence of SEQ ID NO: 21, a V_{L} CDR1 having the amino acid sequence of SEQ ID NO: 28, a V_{L} CDR2 having the amino acid sequence of SEQ ID NO: 35, and a V_{L} CDR3 having the amino acid sequence of SEQ ID NO: 42.

In an embodiment, the monoclonal antibody of claim 1 comprises a V_{H} and a V_{L}. In an embodiment, the V_{H} comprises the amino acid sequence of SEQ ID NO: 43, and the V_{L} comprises the amino acid sequence of SEQ ID NO: 50. In an embodiment, the V_{H} comprises the amino acid sequence of SEQ ID NO: 44, and the V_{L} comprises the amino acid sequence of SEQ ID NO: 51. In an embodiment, the V_{H} comprises the amino acid sequence of SEQ ID NO: 45, and the V_{L} comprises the amino acid sequence of SEQ ID NO: 52. In an embodiment, the V_{H} comprises the amino acid sequence of SEQ ID NO: 46, and the V_{L} comprises the amino acid sequence of SEQ ID NO: 53. In an embodiment, the V_{H} comprises the amino acid sequence of SEQ ID NO: 47, and the V_{L} comprises the amino acid sequence of SEQ ID NO: 54. In an embodiment, the V_{H} comprises the amino acid sequence of SEQ ID NO: 48, and the V_{L} comprises the amino acid sequence of SEQ ID NO: 55. In an embodiment, the V_{H} comprises the amino acid sequence of SEQ ID NO: 49, and the V_{L} comprising the amino acid sequence of SEQ ID NO: 56.

In an embodiment, the monoclonal antibody further comprises a covalently or non-covalently attached conjugate. In an embodiment, the conjugate includes an enzyme, a fluorescence protein, a fluorophore, biotin, or streptavidin. In an embodiment, the enzyme includes HRP. In an embodiment, the monoclonal antibody is a humanized or chimeric antibody.

The disclosure herein also provides a kit for detecting SARS-CoV-2 or a nucleocapsid protein of SARS-CoV-2, and the kit comprises the monoclonal antibody.

Embodiments further provide a method for a rapid test of SARS-CoV-2 infection or fast screening of SARS-CoV-2 carriers. In an embodiment, the method can include mixing a sample with the rabbit mAb against the SARS-CoV-2 NP. In an embodiment, the method is based on a direct ELISA. In an embodiment, the method is based on a capture ELISA. In an embodiment, the method is based on a sandwich ELISA. In an embodiment, the method further includes: adding a secondary antibody comprising a conjugate for detection.

### BRIEF DESCRIPTION OF DRAWINGS

FIG. 1 illustrates a structure of a rabbit mAb against SARS-CoV-2 NP, in accordance with an embodiment.

FIG. 2A illustrates sequence alignment of CDR1s of heavy chains of the rabbit mAb against the nucleocapsid protein of SARS-CoV-2, in accordance with embodiments, including 11B12, 5B4, 1F8, 1G7, 3A7, 6H7, and 5B12.

FIG. 2B illustrates sequence alignment of CDR2s of heavy chains of the rabbit mAb against the nucleocapsid protein of SARS-CoV-2, in accordance with embodiments, including in accordance with embodiments, including 11B12, 5B4, 1F8, 1G7, 3A7, 6H7, and 5B12.

FIG. 2C illustrates sequence alignment of CDR3s of heavy chains of the rabbit mAb against the nucleocapsid protein of SARS-CoV-2, in accordance with embodiments, including 11B12, 5B4, 1F8, 1G7, 3A7, 6H7, and 5B12.

FIG. 3A illustrates sequence alignment of CDR1s of light chains of the rabbit mAb against the nucleocapsid protein of SARS-CoV-2, in accordance with embodiments, including 11B12, 5B4, 1F8, 1G7, 3A7, 6H7, and 5B12.

FIG. 3B illustrates sequence alignment of CDR2s of light chains of the rabbit mAb against the nucleocapsid protein of SARS-CoV-2, in accordance with embodiments, including 11B12, 5B4, 1F8, 1G7, 3A7, 6H7, and 5B12.

FIG. 3C illustrates sequence alignment of CDR3s of light chains of the rabbit mAb against the nucleocapsid protein of SARS-CoV-2, in accordance with embodiments, including 11B12, 5B4, 1F8, 1G7, 3A7, 6H7, and 5B12.

FIG. 4 illustrates sequence alignment of variable regions of heavy chains of the rabbit mAb against the nucleocapsid protein of SARS-CoV-2, in accordance with embodiments, including 11B12, 5B4, 1F8, 1G7, 3A7, 6H7, and 5B12.

FIG. 5 illustrates sequence alignment of variable regions of light chains of the rabbit mAb against the nucleocapsid protein of SARS-CoV-2, in accordance with embodiments, including 11B12, 5B4, 1F8, 1G7, 3A7, 6H7, and 5B12.

FIG. 6 illustrates sequence alignment of heavy chains of Fab fragments of the rabbit mAb against the nucleocapsid protein of SARS-CoV-2, in accordance with embodiments, including 11B12, 5B4, 1F8, 1G7, 3A7, 6H7, and 5B12.

FIG. 7 illustrates sequence alignment of light chains of Fab fragments of the rabbit mAb against the nucleocapsid protein of SARS-CoV-2, in accordance with embodiments, including 11B12, 5B4, 1F8, 1G7, 3A7, 6H7, and 5B12.

FIG. 8 shows the binding curves of 11B12, 5B4, 1F8, 1G7, 3A7, 6H7, and 5B12 to NP in a direct antigen ELISA for detecting the nucleocapsid protein of SARS-CoV-2, in accordance with an embodiment.

FIG. 9 shows cross-reactivity of 11B12, 5B4, 1F8, 1G7, 3A7, 6H7, and 5B12 to nucleocapsid proteins (NP) of SARS-CoV-2, SARS, MERS, hCoV-NL63, in accordance with an embodiment.

FIG. 10 A shows results of sandwich ELISA using 6H7 as the capture antibody and 1G2 as the detection antibody, in accordance with an embodiment.

FIG. 10 B shows results of sandwich ELISA using 6H7 as the capture antibody and 5B12 as the detection antibody, in accordance with an embodiment.

FIG. 10 C shows results of sandwich ELISA using 11B12 as the capture antibody and 5B4 as the detection antibody, in accordance with an embodiment.

FIG. 10 D shows results of sandwich ELISA using 1F8 as the capture antibody and 5B12 as the detection antibody, in accordance with an embodiment.

FIG. 10 E shows results of sandwich ELISA using 5B4 as the capture antibody and 6H7 as the detection antibody, in accordance with an embodiment.

FIG. 10 F shows results of sandwich ELISA using 3A7 as the capture antibody and 1F8 as the detection antibody, in accordance with an embodiment.

### DETAILED DESCRIPTION

The disclosure generally relates to an antibody against SARS-CoV-2. Specifically, the disclosure relates to a rabbit monoclonal antibody (mAb) against the SARS-CoV-2 NP and use thereof.

The term “antibody” herein can be used in the broadest sense and encompasses various antibody structures, including but not limited to a Y-shaped antibody, namely full-length antibody, an antigen-binding portion of the Y-shaped antibody, and a genetic or chemical modification thereof. The antigen-binding portion refers to one or more portions or fragments of the Y-shaped antibody and can retain the ability of the antibody to bind to the SARS-CoV-2 NP specifically.

The term “monoclonal antibody” (mAb) refers to an antibody having a substantially homogeneous population. The individual antibodies of the population are substantially identical, except for possible naturally occurring mutations that may be present in minor amounts. A monoclonal antibody can display a single binding specificity and affinity for a particular epitope on an antigen. In contrast to polyclonal antibodies that typically include different antibodies directed against different epitopes, each monoclonal antibody can target the same or substantially identical epitope on the antigen. The modifier “monoclonal” indicates the character of the antibody as being obtained from a substantially homogeneous population of antibodies, and is not to be construed as requiring the production of the antibody by any particular method. The antibody can be made by various methods, including but limited to, for example, hybridoma method, recombinant DNA methods, phage antibody libraries, and the like.

The terms “mAb against the SARS-CoV-2 NP” and “mAb against the nucleocapsid protein of SARS-CoV-2” are used interchangeably and refer to monoclonal antibodies capable of binding the nucleocapsid protein of SARS-CoV-2 with sufficient affinity so that the antibodies are useful as a detecting, diagnostic, and/or therapeutic agent in targeting SARS-CoV-2. The term “affinity” refers to the strength of the total of non-covalent intermolecular interactions between a single binding site of a molecule (e.g., an antibody) and its binding partner (e.g., an antigen). The intermolecular interactions can include hydrogen bonding, electrostatic interactions, hydrophobic, and Van der Waals forces.

The modifier “rabbit” in the term “rabbit antibody” or “rabbit mAb against the SARS-CoV-2 NP” or the like indicates the complementarity-determining regions (CDRs) of the antibody are derived from rabbit germline immunoglobulin sequences. In an embodiment, the rabbit mAb against the SARS-CoV-2 NP may include antibodies whose CDRs and FRs are derived from rabbit germline immunoglobulin sequences. In an embodiment, the rabbit antibody or rabbit mAb against the nucleocapsid protein of SARS-CoV-2 can encompass antibodies whose CDRs are derived from rabbit germline immunoglobulin sequences. In an embodiment, the rabbit mAb against the SARS-CoV-2 NP may encompass antibodies whose CDRs are derived from rabbit germline immunoglobulin sequences and whose framework regions (FRs) are derived from germline immunoglobulin sequences of another mammalian species, such as mouse or human. The term “rabbit mAb against the SARS-CoV-2 NP” may also encompass antibodies containing amino acid residues not encoded by rabbit germline immunoglobulin sequences, e.g., mutations introduced by random or site-specific mutagenesis in vitro or by somatic mutation in vivo. However, the term “rabbit mAb against the SARS-CoV-2 NP” is not intended to include antibodies whose CDRs are derived from the germline of another mammalian species, such as a mouse.

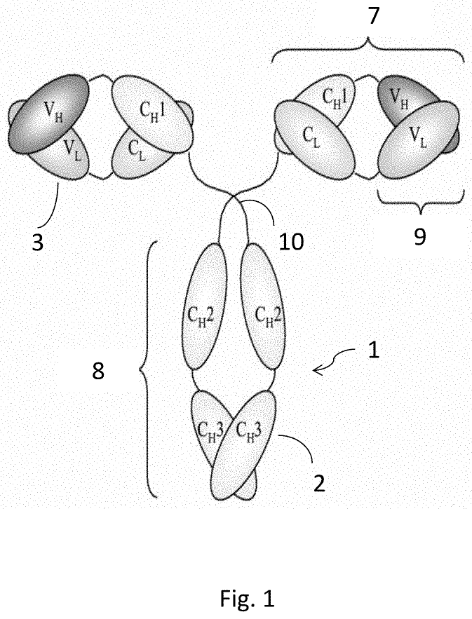

In an embodiment, the rabbit mAb against the SARS-CoV-2 NP can be a Y-shaped antibody. Referring to FIG. 1, FIG. 1 illustrates a Y-shaped structure of a rabbit mAb against the SARS-CoV-2 NP in accordance with an embodiment. In an embodiment, the rabbit mAb against the SARS-CoV-2 NP can include two pairs of heavy chain 2 and light chain 3. The heavy chain 2 can include one variable region (V_{H}) and one or more constant regions (C_{H}s). In an embodiment, the heavy chain 2 can include one V_{H} and three C_{H}s, namely C_{H}1, C_{H}2, and C_{H}3. The V_{H} is closer to the N-terminus of the heavy chain as compared to the three C_{H}s. The V_{H} can exhibit higher variability in the amino acid sequence as compared to the C_{H}s. The V_{H} can differ between different antibodies and can be specific to each antibody. The amino acid sequences of the C_{H}s can be identical across all antibodies of the same isotype (class) but differ between isotypes. The term “isotype” refers to the antibody class (e.g., IgG) encoded by the heavy-chain constant-region genes. Mammalian antibodies can include five types of heavy chains: γ, δ, α, μ, and ε. They define classes of antibodies: IgG, IgD, IgA, IgM, and IgE, respectively.

The light chain 3 can be a small polypeptide subunit relative to the heavy chain 2. The light chain 3 can include one variable region (V_{L}) and one constant (C_{L}) region. The V_{L} is generally is the N-terminus portion of the light chain 3 and exhibits higher variability in amino acid sequence than the C_{L}. The V_{L} can differ between different antibodies and be specific to each antibody in amino acid sequence.

In an embodiment, the variable regions, V_{H} and V_{L}, are responsible for recognizing and binding the NP. In an embodiment, C_{H}s and C_{L} do not directly contact residues of the nucleocapsid protein.

The two pairs of heavy chain 2 and light chain 3 can form a Y-shaped structure that includes two Fab (Fragment antigen-binding) fragments 7, one Fc (Fragment crystallizable) fragment 8, and hinge regions 10. The two Fab fragments 7 look like the two arms of the “Y”, and the Fc fragment 8 looks like the base of the “Y”. The hinge regions 10 connect the Fc fragment 8 with the two Fab fragments 7.

Each of the Fab fragments 7 can comprise the V_{H} and C_{H}1 from the heavy chain 2 and the V_{L} and C_{L} from the light chain 3. The Fab fragment 7 contains a variable fragment (Fv fragment) 9 formed of the V_{L} and V_{H}. The Fv fragment 9 accommodates the antigen-binding site, namely paratope. The paratope can be at the tip of the arm of the Y-shaped rabbit mAb 1.

Each of the variable regions, V_{H} and V_{L}, can include complementarity-determining regions (CDRs) and framework regions (FRs). The CDRs determine the specificity and binding affinity of the Y-shaped rabbit mAb 1. The CDRs contain the antigen-contacting residues and are responsible for the ability of the rabbit mAb 1 to recognize and contact the NP. The Y-shaped rabbit mAb 1 can include six (6) CDRs, three of which are in the V_{H}, namely V_{H} CDR1, V_{H} CDR2, and V_{H} CDR3, and the other three of which are in the V_{L}, namely V_{L} CDR1, V_{L} CDR3, and V_{L} CDR3.

The CDRs of V_{H} and V_{L} can each be separated by the FRs. The FRs are conserved regions in sequence structures. The FRs can generally act as a scaffold so that the CDRs can adopt three-dimensional structures capable of directly contacting the antigen, i.e., the NP. The three-dimensional structure of the FRs can be conserved across different antibodies. In an embodiment, the CDRs of the Y-shaped rabbit mAb 1 can be grafted into FRs of another antibody from other species while retaining their ability to bind the nucleocapsid protein, forming a mosaic antibody. In an embodiment, the CDRs of the Y-shaped rabbit mAb 1 are grafted into FRs of a human antibody, forming a humanized antibody against the NP.

The Fc fragment 8 can be formed of C_{H}2 and C_{H}3 from the two heavy chains 2. In an embodiment, the Fc fragment 8 can include three constant domains. As the Fc fragment 8 can be composed of the constant domains of the heavy chains, the classes of the heavy chains can be used to categorize the antibody. The Fc fragment 8 of the Y-shaped rabbit mAb 1 generally does not involve binding the antigen. In an embodiment, the Fc fragment 8 can play a role in modulating immune cell activity, for example, by binding to a specific class of Fc receptors or other immune molecules such as complement proteins. In an embodiment, the Fc fragment 8 can play a role in generating an appropriate immune response when the CDRs bind to the antigen. In an embodiment, the Fc fragment 8 can mediate different physiological effects that can include but not limited to recognition of opsonized particles when binding to FcγR, degranulation of mast cells, basophils, and eosinophils when binding to Fcε receptors, lysis of cells or complement-dependent cytotoxicity when binding to complement proteins, antibody-dependent cellular cytotoxicity (ADCC), antibody-dependent cell phagocytosis (ADCP), interaction with the neonatal Fc receptor (FcRn) to slow down antibody degradation and extend its serum half-life.

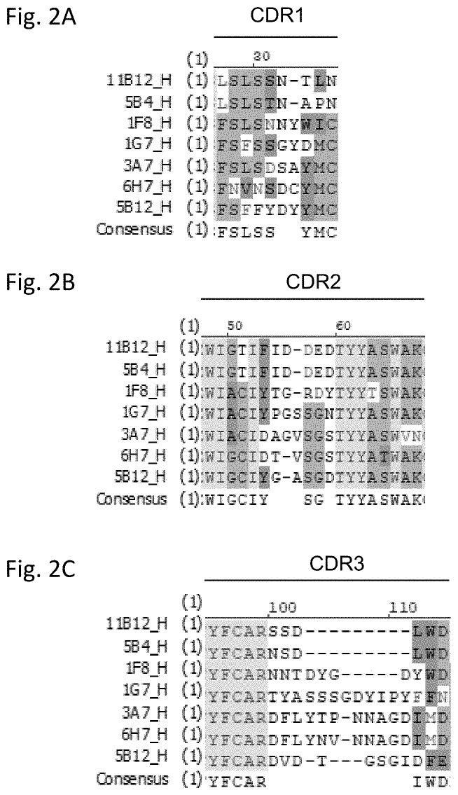

FIGS. 2A, 2B, and 2C respectively show sequence alignments of CDR1, CDR2, and CDR3 of the heavy chain variable regions (V_{H}) of the rabbit mAb against the nucleocapsid protein of SARS-CoV-2, in accordance with embodiments, including 11B12, 5B4, 1F8, 1G7, 3A7, 6H7, and 5B12.

Referring to FIG. 2A, the V_{H} CDR1s of 11B12, 5B4, 1F8, 1G7, 3A7, 6H7, and 5B12 can have a length of about 8-10 amino acids. The amino acid sequence of the V_{H} CDR1 of 11B12 can include or consist of LSLSSNTLN (SEQ ID NO: 1). The amino acid sequence of the V_{H} CDR1 of 5B4 can include or consist of LSLSTNAPN (SEQ ID NO: 2). The amino acid sequence of the V_{H} CDR1 of 1F8 can include or consist of FSLSNNYWIC (SEQ ID NO: 3). The amino acid sequence of the V_{H} CDR1 of 1G7 can include or consist of FSFSSGYDMC (SEQ ID NO: 4). The amino acid sequence of the V_{H} CDR1 of 3A7 can include or consist of FSLSDSAYMC (SEQ ID NO: 5). The amino acid sequence of V_{H} CDR1 of 6H7 can include or consist of FNVNSDCYMC (SEQ ID NO: 6). The amino acid sequence of V_{H} CDR1 of 5B12 can include or consist of FSFFYDYYMC (SEQ ID NO: 7). The V_{H} CDR1s of 11B12, 5B4, 1F8, 1G7, 3A7, 6H7, and 5B12 have a consensus sequence formula of FSLSS[ ][ ]YMC. The pair of square brackets “[ ]” represents a single position in a protein sequence.

Referring to FIG. 2B, the V_{H} CDR2s of 11B12, 5B4, 1F8, 1G7, 3A7, 6H7, and 5B12 can have a length of about 19-20 amino acids. The amino acid sequence of the V_{H} CDR2 of 11B12 can include or consist of WIGTIFIDDEDTYYASWAK (SEQ ID NO: 8). The amino acid sequence of the V_{H} CDR2 of 5B4 can include or consist of WIGTIFIDDEDTYYASWAK (SEQ ID NO: 9). The amino acid sequence of the V_{H} CDR2 of 1F8 can include or consist of WIACIYTGRDYTYYTSWAK (SEQ ID NO: 10). The amino acid sequence of the V_{H} CDR2 of 1G7 can include or consist of WIACIYPGSSGNTYYASWAK (SEQ ID NO: 11). The amino acid sequence of the V_{H} CDR2 of 3A7 can include or consist of WIACIDAGVSGSTYYASWVN (SEQ ID NO: 12). The amino acid sequence of V_{H} CDR2 of 6H7 can include or consist of WIGCIDTVSGSTYYATWAK (SEQ ID NO: 13). The amino acid sequence of V_{H} CDR2 of 5B12 can include or consist of WIGCIYGASGDTYYASWAK (SEQ ID NO: 14). The V_{H} CDR2s of 11B12, 5B4, 1F8, 1G7, 3A7, 6H7, and 5B12 have a consensus sequence formula of WIGCIY[ ][ ][ ]SG[ ]TYYASWAK.

Referring to FIG. 2C, the V_{H} CDR3s of 11B12, 5B4, 1F8, 1G7, 3A7, 6H7, and 5B12 can have a length of about 11-20 amino acids. The amino acid sequence of the V_{H} CDR3 of 11B12 can include or consist of YFCARSSDLWD (SEQ ID NO: 15). The amino acid sequence of the V_{H} CDR3 of 5B4 can include or consist of YFCARNSDLWD (SEQ ID NO: 16). The amino acid sequence of the V_{H} CDR3 of 1F8 can include or consist of YFCARNNTDYGDYWD (SEQ ID NO: 17). The amino acid sequence of the V_{H} CDR3 of 1G7 can include or consist of YFCARTYASSSGDYIPYFFN (SEQ ID NO: 18). The amino acid sequence of the V_{H} CDR3 of 3A7 can include or consist of YFCARDFLYTPNNAGDIMD (SEQ ID NO: 19). The amino acid sequence of V_{H} CDR3 of 6H7 can include or consist of YFCARDFLYNVNNAGDIMD (SEQ ID NO: 20). The amino acid sequence of V_{H} CDR3 of 5B12 can include or consist of YFCARDVDTGSGIDFE (SEQ ID NO: 21). The V_{H} CDR3s of 11B12, 5B4, 1F8, 1G7, 3A7, 6H7, and 5B12 have a consensus sequence formula of YFCAR[ ][ ][ ][ ][ ][ ][ ][ ][ ][ ][ ][ ]IWD.

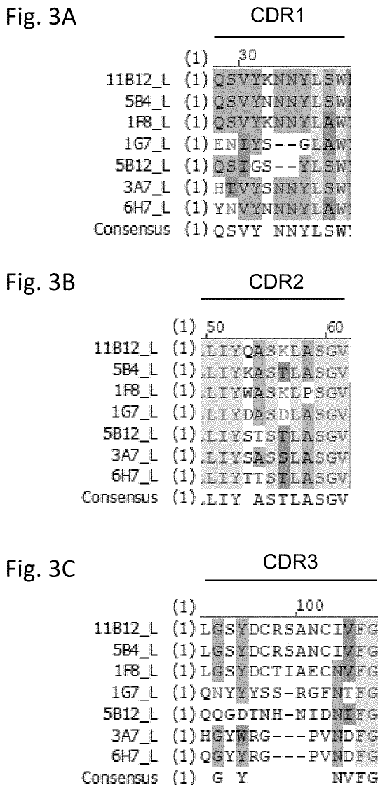

FIGS. 3A, 3B, and 3C respectively show sequence alignments of CDR1, CDR2, and CDR3 of the light chain variable regions (V_{L}) of the rabbit mAb against the SARS-CoV-2 NP, in accordance with embodiments, including 11B12, 5B4, 1F8, 1G7, 3A7, 6H7, and 5B12.

Referring to FIG. 3A, the V_{L}CDR1s of 11B12, 5B4, 1F8, 1G7, 3A7, 6H7, and 5B12 can have a length of about 9-11 amino acids. The amino acid sequence of the V_{L} CDR1 of 11B12 can include or consist of QSVYKNNYLSW (SEQ ID NO: 22). The amino acid sequence of the V_{L} CDR1 of 5B4 can include or consist of QSVYNNNYLSW (SEQ ID NO: 23). The amino acid sequence of the V_{L} CDR1 of 1F8 can include or consist of QSVYKNNYLAW (SEQ ID NO: 24). The amino acid sequence of the V_{L} CDR1 of 1G7 can include or consist of ENIYSGLAW (SEQ ID NO: 25). The amino acid sequence of the V_{L} CDR1 of 3A7 can include or consist of HTVYSNNYLSW (SEQ ID NO: 26). The amino acid sequence of V_{L} CDR1 of 6H7 can include or consist of YNVYNNNYLAW (SEQ ID NO: 27). The amino acid sequence of V_{L} CDR1 of 5B12 can include or consist of QSIGSYLSW (SEQ ID NO: 28). The V_{L} CDR1s of 11B12, 5B4, 1F8, 1G7, 3A7, 6H7, and 5B12 have a sequence consensus formula of QSVY[ ]NNYLSW. The pair of square brackets “[ ]” represents a single position in a protein sequence.

Referring to FIG. 3B, the V_{L} CDR2s of 11B12, 5B4, 1F8, 1G7, 3A7, 6H7, and 5B12 can have a length of about 12 amino acids. The amino acid sequence of the V_{L} CDR2 of 11B12 can include or consist of LIYQASKLASGV (SEQ ID NO: 29). The amino acid sequence of the V_{L} CDR2 of 5B4 can include or consist of LIYKASTLASGV (SEQ ID NO: 30). The amino acid sequence of the V_{L} CDR2 of 1F8 can include or consist of LIYWASKLPSGV (SEQ ID NO: 31). The amino acid sequence of the V_{L} CDR2 of 1G7 can include or consist of LIYDASDLASGV (SEQ ID NO: 32). The amino acid sequence of the V_{L} CDR2 of 3A7 can include or consist of LIYSASSLASGV (SEQ ID NO: 33). The amino acid sequence of V_{L} CDR2 of 6H7 can include or consist of LIYTTSTLASGV (SEQ ID NO: 34). The amino acid sequence of V_{L} CDR2 of 5B12 can include or consist of LIYSTSTLASGV (SEQ ID NO: 35). The V_{L} CDR2s of 11B12, 5B4, 1F8, 1G7, 3A7, 6H7, and 5B12 have a consensus sequence formula of LIY[ ]ASTLASGV.

Referring to FIG. 3C, the V_{L} CDR3s of 11B12, 5B4, 1F8, 1G7, 3A7, 6H7, and 5B12 can have a length of about 12-15 amino acids. The amino acid sequence of the V_{L} CDR3 of 11B12 can include or con LGSYDCRSANCIVFG (SEQ ID NO: 36). The amino acid sequence of the V_{L} CDR3 of 5B4 can include or consist of LGSYDCRSANCIVFG (SEQ ID NO: 37). The amino acid sequence of the V_{L} CDR3 of 1F8 can include or consist of LGSYDCTIAECNVFG (SEQ ID NO: 38). The amino acid sequence of the V_{L} CDR3 of 1G7 can include or consist of QNYYYSSRGFNTFG (SEQ ID NO: 39). The amino acid sequence of the V_{L} CDR3 of 3A7 can include or consist of HGYWRGPVNDFG (SEQ ID NO: 40). The amino acid sequence of V_{L} CDR3 of 6H7 can include or consist of QGYYRGPVNDFG (SEQ ID NO: 41). The amino acid sequence of V_{L} CDR3 of 5B12 can include or consist of QQGDTNHNIDNIFG (SEQ ID NO: 42). The V_{L} CDR3s of 11B12, 5B4, 1F8, 1G7, 3A7, 6H7, and 5B12 have a consensus sequence formula of [ ]G[ ]Y[ ][ ][ ][ ][ ][ ][ ]NVFG.

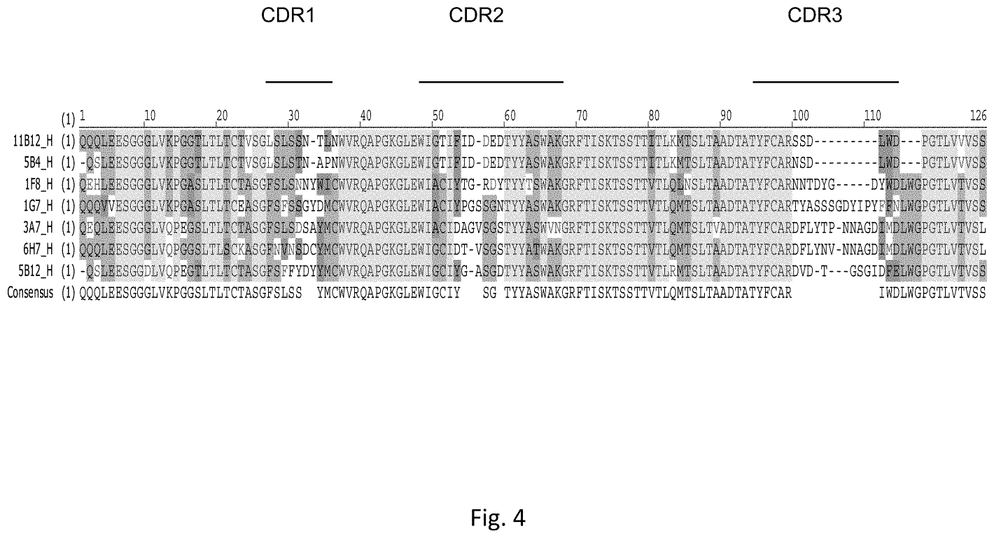

Referring to FIG. 4, FIG. 4 shows a sequence alignment of variable regions of heavy chains of the rabbit mAb against the nucleocapsid protein of SARS-CoV-2, in accordance with embodiments, including 11B12, 5B4, 1F8, 1G7, 3A7, 6H7, and 5B12.

The amino acid sequence of the V_{H} of 11B12 can include or consist of

(SEQ ID NO: 43)
QQQLEESGGGLVKPGGTLTLTCTVSGLSLSSNTLNWVRQAPGKGLEWIG
TIFIDDEDTYYASWAKGRFTISKTSSTTITLKMTSLTAADTATYFCARS
SDLWDPGTLVVVSS.

The amino acid sequence of the V_{H} of 5B4 can include or consist of

(SEQ ID NO: 44)
QSLEESGGGLVKPGGTLTLTCTVSGLSLSTNAPNWVRQAPGKGLEWIGT
IFIDDEDTYYASWAKGRFTISKTSSTTITLKMTSLTAADTATYFCARNS
DLWDPGTLVVVSS.

The amino acid sequence of the V_{H} of 1F8 can include or consist of

(SEQ ID NO: 45)
QEHLEESGGGLVKPGASLTLTCTASGFSLSNNYWICWVRQAPGKGLEWI
ACIYTGRDYTYYTSWAKGRFTISKTSSTTVTLQLNSLTAADTATYFCAR
NNTDYGDYWDLWGPGTLVTVSS.

The amino acid sequence of the V_{H} of 1G7 can include or consist of

(SEQ ID NO: 46)
QQQVVESGGGLVKPGASLTLTCEASGFSFSSGYDMCWVRQAPGKGLEWI
ACIYPGSSGNTYYASWAKGRFTISKTSSTTVTLQMTSLTAADTATYFCA
RTYASSSGDYIPYFFNLWGPGTLVTVSS.

The amino acid sequence of the V_{H} of 3A7 can include or consist of

(SEQ ID NO: 47)
QEQLEESGGGLVQPEGSLTLTCTASGFSLSDSAYMCWVRQAPGKGLEW
IACIDAGVSGSTYYASWVNGRFTISKTSSTTVTLQMTSLTVADTATYF
CARDFLYTPNNAGDIMDLWGPGTLVTVSL.

The amino acid sequence of the V_{H} of 6H7 can include or consist of

(SEQ ID NO: 48)
QQQLEESGGGLVQPGGSLTLSCKASGFNVNSDCYMCWVRQAPGKGLEW
IGCIDTVSGSTYYATWAKGRFTISKTSSTTVTLQMTSLTAADTATYFC
ARDFLYNVNNAGDIMDLWGPGTLVTVSL.

The amino acid sequence of the V_{H} of 5B12 can include or consist of

(SEQ ID NO: 49)
QSLEESGGDLVQPEGTLTLTCTASGFSFFYDYYMCWVRQAPGKGLEWI
GCIYGASGDTYYASWAKGRFTISKTSSTTVTLRWITSLTAADTATYFC
ARDVDTGSGIDFELWGPGTLVTVSS.

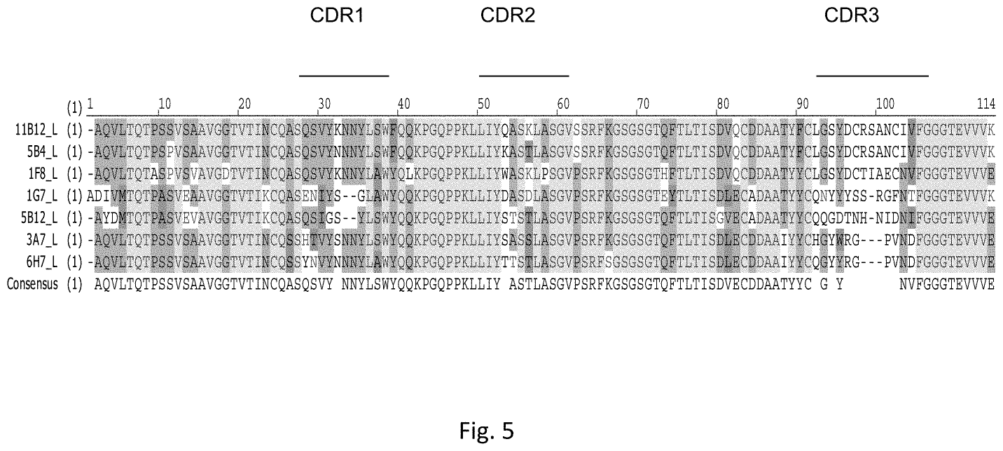

Referring to FIG. 5, FIG. 5 shows a sequence alignment of variable regions of light chains of the rabbit mAb against the nucleocapsid protein of SARS-CoV-2, in accordance with embodiments including 11B12, 5B4, 1F8, 1G7, 3A7, 6H7, and 5B12.

The amino acid sequence of the V_{L} of 11B12 can include

(SEQ ID NO: 50)
AQVLTQTPSSVSAAVGGTVTINCQASQSVYKNNYLSWFQQKPGQPPKL
LIYQASKLASGVSSRFKGSGSGTQFTLTISDVQCDDAATYFCLGSYDC
RSANCIVFGGGTEVVVK.

The amino acid sequence of the V_{L} of 5B4 can include

(SEQ ID NO: 51)
AQVLTQTPSPVSAAVGGTVTINCQASQSVYNNNYLSWFQQKPGQPPKW
YKASTLASGVSSRFKGSGSGTQFTLTISDVQCDDAATYFCLGSYDCRS
ANCIVFGGGTEVVVK.

The amino acid sequence of the V_{L} of 1F8 can include

(SEQ ID NO: 52)
AQVLTQTASPVSVAVGDTVTINCQASQSVYKNNYLAWYQLKPGQPPK
WYWGTKLPSGVPSRFKGSGSGTHFTLTISDVQCDDAATYYCLGSYDC
TIAECNVFGGGTEVVVE.

The amino acid sequence of the V_{L} of 1G7 can include

(SEQ ID NO: 53)
ADIVMTQTPASVEAAVGGTVTIKCQASENIYSGLAWYQQKPGQPPKL
LIYDASDLASGVPSRFKGSGSGTEYTLTISDLECADAATYYCQNYYY
SSRGFNTFGGGTEVVVK.

The amino acid sequence of the V_{L} of 3A7 can include

(SEQ ID NO: 54)
AQVLTQTPSSVSAAVGGTVTINCQSSHTVYSNNYLSWYQQKPGQPPK
LLIYSASSLASGVPSRFKGSGSGTQFTLTISDLECDDAAIYYCHGYW
RGPVNDFGGGTEVVVE.

The amino acid sequence of the V_{L} of 6H7 can include

(SEQ ID NO: 55)
AQVLTQTPSSVSAAVGGTVTINCQSSYNVYNNNYLAWYQQKPGQPPK
LLIYTTSTLASGVPSRFSGSGSGTQFTLTISDLECDDAAIYYCQGYY
RGPVNDFGGGTEVVVE.

The amino acid sequence of the V_{L} of 5B12 can include

(SEQ ID NO: 56)
AYDMTQTPASVEVAVGGTVTIKCQASQSIGSYLSW
YQQKPGQPPKLLIYSTSTLASGVPSRFKGSGSGTQ
FTLTISGVECADAATYYCQQGDTNHNIDNIFGGGT
EVVVE.

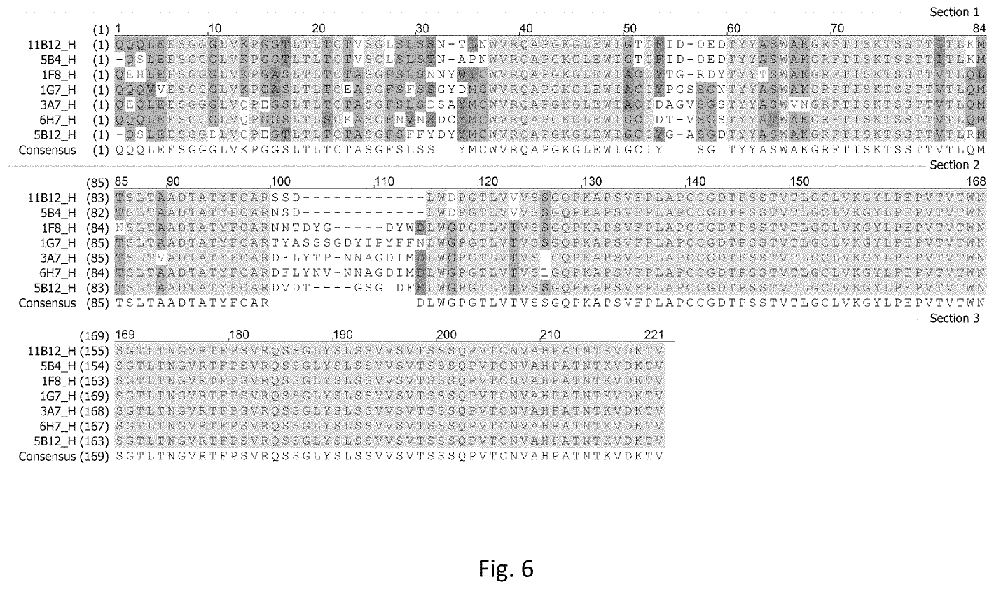

Referring to FIG. 6, FIG. 6 shows a sequence alignment of heavy chains of Fab fragments of the rabbit mAb against the nucleocapsid protein of SARS-CoV-2, in accordance with embodiments including 11B12, 5B4, 1F8, 1G7, 3A7, 6H7, and 5B12.

The amino acid sequence of the heavy chain of the Fab fragment of 11B12 can include or consist of

(SEQ ID NO: 57)
QQQLEESGGGLVKPGGTLTLTCTVSGLSLSSNTLN
WVRQAPGKGLEWIGTIFIDDEDTYYASWAKGRFTI
SKTSSTTITLKMTSLTAADTATYFCARSSDLWDPG
TLVVVSSGQPKAPSVFPLAPCCGDTPSSTVTLGCL
VKGYLPEPVTVTWNSGTLTNGVRTFPSVRQSSGLY
SLSSVVSVTSSSQPVTCNVAHPATNTKVDKTV.

The amino acid sequence of the heavy chain of the Fab fragment of 5B4 can include or consist of

(SEQ ID NO: 58)
QSLEESGGGLVKPGGTLTLTCTVSGLSLSTNAPNW
VRQAPGKGLEWIGTIFIDDEDTYYASWAKGRFTIS
KTSSTTITLKMTSLTAADTATYFCARNSDLWDPGT
LVVVSSGQPKAPSVFPLAPCCGDTPSSTVTLGCLV
KGYLPEPVTVTWNSGTLTNGVRTFPSVRQSSGLYS
LSSVVSVTSSSQPVTCNVAHPATNTKVDKTV.

The amino acid sequence of the heavy chain of the Fab fragment of 1F8 can include or consist of

(SEQ ID NO: 59)
QEHLEESGGGLVKPGASLTLTCTASGFSLSNNYWI
CWVRQAPGKGLEWIACIYTGRDYTYYTSWAKGRFT
ISKTSSTTVTLQLNSLTAADTATYFCARNNTDYGD
YWDLWGPGTLVTVSSGQPKAPSVFPLAPCCGDTPS
STVTLGCLVKGYLPEPVTVTWNSGTLTNGVRTFPS
VRQSSGLYSLSSVVSVTSSSQPVTCNVAHPATNTK
VDKTV.

The amino acid sequence of the heavy chain of the Fab fragment of 1G7 can include or consist of

(SEQ ID NO: 60)
QQQVVESGGGLVKPGASLTLTCEASGFSFSSGYDM
CWVRQAPGKGLEWIACIYPGSSGNTYYASWAKGRF
TISKTSSTTVTLQMTSLTAADTATYFCARTYASSS
GDYIPYFFNLWGPGTLVTVSSGQPKAPSVFPLAPC
CGDTPSSTVTLGCLVKGYLPEPVTVTWNSGTLTNG
VRTFPSVRQSSGLYSLSSVVSVTSSSQPVTCNVAH
PATNTKVDKTV.

The amino acid sequence of the heavy chain of the Fab fragment of 3A7 can include or consist of

(SEQ ID NO: 61)
QEQLEESGGGLVQPEGSLTLTCTASGFSLSDSAYM
CWVRQAPGKGLEWIACIDAGVSGSTYYASWVNGRF
TISKTSSTTVTLQMTSLTVADTATYFCARDFLYTP
NNAGDIMDLWGPGTLVTVSLGQPKAPSVFPLAPCC
GDTPSSTVTLGCLVKGYLPEPVTVTWNSGTLTNGV
RTFPSVRQSSGLYSLSSVVSVTSSSQPVTCNVAHP
ATNTKVDKTV.

The amino acid sequence of the heavy chain of the Fab fragment of 6H7 can include of consist of

(SEQ ID NO: 62)
QQQLEESGGGLVQPGGSLTLSCKASGFNVNSDCYM
CWVRQAPGKGLEWIGCIDTVSGSTYYATWAKGRFT
ISKTSSTTVTLQMTSLTAADTATYFCARDFLYNVN
NAGDIMDLWGPGTLVTVSLGQPKAPSVFPLAPCCG
DTPSSTVTLGCLVKGYLPEPVTVTWNSGTLTNGVR
TFPSVRQSSGLYSLSSVVSVTSSSQPVTCNVAHPA
TNTKVDKTV.

The amino acid sequence of the heavy chain of the Fab fragment of 5B12 can include or consist of

(SEQ ID NO: 63)
QSLEESGGDLVQPEGTLTLTCTASGFSFFYDYYMC
WVRQAPGKGLEWIGCIYGASGDTYYASWAKGRFTI
SKTSSTTVTLRWITSLTAADTATYFCARDVDTGSG
IDFELWGPGTLVTVSSGQPKAPSVFPLAPCCGDTP
SSTVTLGCLVKGYLPEPVTVTWNSGTLTNGVRTFP
SVRQSSGLYSLSSVVSVTSSSQPVTCNVAHPATNT
KVDKTV.

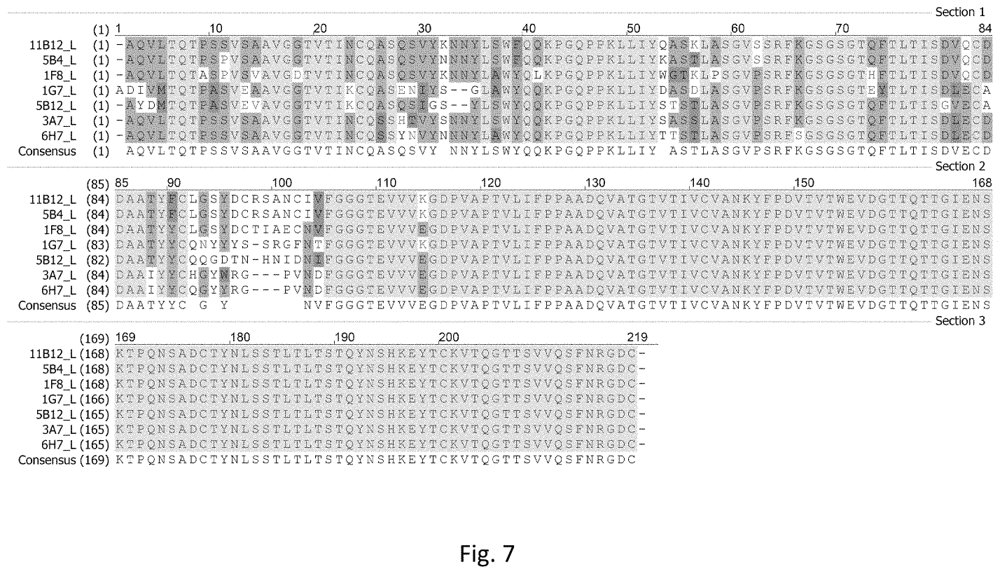

Referring to FIG. 7, FIG. 7 shows a sequence alignment of light chains of Fab fragments of the rabbit mAb against the nucleocapsid protein of SARS-CoV-2, in accordance with embodiments including 11B12, 5B4, 1F8, 1G7, 3A7, 6H7, and 5B12.

The amino acid sequence of the light chain of the Fab fragment of 11B12 can include or consist of

(SEQ ID NO: 64)
AQVLTQTPSSVSAAVGGTVTINCQASQSVYKNNYL
SWFQQKPGQPPKLLIYQASKLASGVSSRFKGSGSG
TQFTLTISDVQCDDAATYFCLGSYDCRSANCIVFG
GGTEVVVKGDPVAPTVLIFPPAADQVATGTVTIVC
VANKYFPDVTVTWEVDGTTQTTGIENSKTPQNSAD
CTYNLSSTLTLTSTQYNSHKEYTCKVTQGTTSVVQ
SFNRGDC.

The amino acid sequence of the light chain of the Fab fragment of 5B4 can include or consist of

(SEQ ID NO: 65)
AQVLTQTPSPVSAAVGGTVTINCQASQSVYNNNYL
SWFQQKPGQPPKWYKASTLASGVSSRFKGSGSGTQ
FTLTISDVQCDDAATYFCLGSYDCRSANCIVFGGG
TEVVVKGDPVAPTVLIFPPAADQVATGTVTIVCVA
NKYFPDVTVTWEVDGTTQTTGIENSKTPQNSADCT
YNLSSTLTLTSTQYNSHKEYTCKVTQGTTSVVQSF
NRGDC.

The amino acid sequence of the light chain of the Fab fragment of 1F8 can include or consist of

(SEQ ID NO: 66)
AQVLTQTASPVSVAVGDTVTINCQASQSVYKNNYL
AWYQLKPGQPPKWYWGTKLPSGVPSRFKGSGSGTH
FTLTISDVQCDDAATYYCLGSYDCTIAECNVFGGG
TEVVVEGDPVAPTVLIFPPAADQVATGTVTIVCVA
NKYFPDVTVTWEVDGTTQTTGIENSKTPQNSADCT
YNLSSTLTLTSTQYNSHKEYTCKVTQGTTSVVQSF
NRGDC.

The amino acid sequence of the light chain of the Fab fragment of 1G7 can include or consist of

(SEQ ID NO: 67)
ADIVMTQTPASVEAAVGGTVTIKCQASENIYSGLA
WYQQKPGQPPKLLIYDASDLASGVPSRFKGSGSGT
EYTLTISDLECADAATYYCQNYYYSSRGFNTFGGG
TEVVVKGDPVAPTVLIFPPAADQVATGTVTIVCVA
NKYFPDVTVTWEVDGTTQTTGIENSKTPQNSADCT
YNLSSTLTLTSTQYNSHKEYTCKVTQGTTSVVQSF
NRGDC.

The amino acid sequence of the light chain of the Fab fragment of 3A7 can include or consist of

(SEQ ID NO: 68)
AQVLTQTPSSVSAAVGGTVTINCQSSHTVYSNNYL
SWYQQKPGQPPKLLIYSASSLASGVPSRFKGSGSG
TQFTLTISDLECDDAAIYYCHGYWRGPVNDFGGGT
EVVVEGDPVAPTVLIFPPAADQVATGTVTIVCVAN
KYFPDVTVTWEVDGTTQTTGIENSKTPQNSADCTY
NLSSTLTLTSTQYNSHKEYTCKVTQGTTSVVQSFN
RGDC.

The amino acid sequence of the light chain of the Fab fragment of 6H7 can include or consist of

(SEQ ID NO: 69)
AQVLTQTPSSVSAAVGGTVTINCQSSYNVYNNNYL
AWYQQKPGQPPKLLIYTTSTLASGVPSRFSGSGSG
TQFTLTISDLECDDAAIYYCQGYYRGPVNDFGGGT
EVVVEGDPVAPTVLIFPPAADQVATGTVTIVCVAN
KYFPDVTVTWEVDGTTQTTGIENSKTPQNSADCTY
NLSSTLTLTSTQYNSHKEYTCKVTQGTTSVVQSFN
RGDC.

The amino acid sequence of the light chain of the Fab fragment of 5B12 can include or consist of

(SEQ ID NO: 70)
AYDMTQTPASVEVAVGGTVTIKCQASQSIGSYLSW
YQQKPGQPPKLLIYSTSTLASGVPSRFKGSGSGTQ
FTLTISGVECADAATYYCQQGDTNHNIDNIFGGGT
EVVVEGDPVAPTVLIFPPAADQVATGTVTIVCVAN
KYFPDVTVTWEVDGTTQTTGIENSKTPQNSADCTY
NLSSTLTLTSTQYNSHKEYTCKVTQGTTSVVQSFN
RGDC.

The rabbit mAb against the SARS-CoV-2 NP also be an antigen-binding portion of the Y-shaped antibodies disclosed herein. In an embodiment, the rabbit mAb against the SARS-CoV-2 NP can be the Fab fragment 7—a monovalent fragment formed of the V_{L}, V_{H}, C_{L}, and C_{H1} domains. In an embodiment, the rabbit mAb against the SARS-CoV-2 NP can be an F(ab′)₂ fragment that is a bivalent fragment including the two Fab fragments 7 linked by, e.g., a disulfide bridge of the hinge region 10. In an embodiment, the rabbit mAb against the SARS-CoV-2 NP can be an Fd fragment formed of the V_{H} and C_{H1} domains. In an embodiment, the rabbit mAb against the SARS-CoV-2 NP can be the Fv fragment 9 formed of the V_{L} and V_{H} domains. In an embodiment, the rabbit mAb against the SARS-CoV-2 NP can be an isolated complementarity-determining region.

The rabbit mAb against the SARS-CoV-2 NP can also encompass structures derived from the embodiments or their antigen-binding portions by genetic modification. Different genetically modified antibody structures can be generated, including but not being limited to humanized antibodies, chimeric antibodies, etc. In an embodiment, the rabbit mAb against the SARS-CoV-2 NP can be a humanized antibody, as its protein sequence has been modified to increase its similarity to antibody variants produced naturally in humans. The protein sequence of a humanized antibody can be essentially identical to that of a human variant, despite the rabbit origin of some of its CDRs that are essential to the ability of the antibody to bind to SARS-CoV-2 NP. In an embodiment, a humanized antibody can be created by inserting CDRs of a non-human antibody, e.g., a rabbit antibody, into a human antibody scaffold. In an embodiment, the rabbit mAb against the SARS-CoV-2 NP can be a chimeric antibody. In an embodiment, the chimeric antibody can be an antibody made by transplanting variable regions of the heavy and light chains from the Y-shaped antibodies herein onto constant regions from another species such as a human. In an embodiment, the chimeric antibody is an antibody made by fusing Fab of one of the rabbit mAb against the SARS-CoV-2 NP s disclosed herein with Fc of a human antibody. In an embodiment, the rabbit mAb against the SARS-CoV-2 NP can be a single-chain Fv (scFv). Although the two domains of the Fv fragment, namely V_{L} and V_{H}, are coded by separate genes, they can be joined, using recombinant methods, by a synthetic linker to form the scFv. In an embodiment, the genetic modification can be performed in accordance with methods known in the art, and the genetically modified antibody structures can be screened in the same manner as the full-length antibodies.

The antibody disclosed herein can also encompass structures derived from the embodiments disclosed herein and their antigen-binding portions by chemical modification. In an embodiment, the chemical modification can be a chemical crosslinking. In an embodiment, one or more conjugates can be covalently linked to or non-covalently attached to the antibody. In an embodiment, the conjugate can be a molecular label covalently attached to the antibody to facilitate the detection of its antigens. The conjugates can be any suitable small molecules. The small molecule can include but is not limited to, for example, biotin, streptavidin, and/or fluorescent dye. The fluorescent dye can be any suitable fluorescent dye, including but not limited to Alexa fluors, aminomethylcoumarin (AMCA), Atto dyes, cyanine dyes, DyLight fluors, FITC, FluoProbes 647H, Rhodamine, and Texas Red. The Alexa fluors include but are not limited to Alexa Fluor 488, Alexa Fluor 555, Alexa Fluor 568, Alexa Fluor 594, Alexa Fluor 647, and Alexa Fluor 700. The Atto dyes can include but are not limited to Atto 390, Atto 488, Atto 565, Atto 633, and Atto 700. The cyanine dyes can include but are not limited to Cy3, Cy5, and Cy5.5. The DyLight dyes can include but not limited to DyLight 350, DyLight 405, DyLight 488, DyLight 550, DyLight 594, DyLight 633, DyLight 650, DyLight 680, DyLight 755, and DyLight 800. In an embodiment, the conjugates can be a tandem dye with two covalently attached fluorescence molecules. In an embodiment, one of the fluorescence molecules serves as a donor, and the other serves as an acceptor. In an embodiment, the donor and the acceptor can behave as a unique fluorophore with the donor's excitation properties and the acceptor's emission properties. The tandem dye can include but is not limited to Allophycocyanin-Cy5.5, Allophycocyanin-Cy 7, PE-Atto 594, PE-Cy 5, PE-Cy 5.5, PE-Cy 7, PE-Texas Red, PE-Alexa Fluor 647, PE-Alexa Fluor 700, PE-Alexa Fluor 750, APC-Alexa Fluor 750, and PerCP-Cy5.5.

The conjugates can also be large molecules. In an embodiment, the large molecule can be an enzyme. The enzyme can include but is not limited to alkaline phosphatase (AP), glucose oxidase (Gox), Horseradish peroxidase (HRP). In an embodiment, the large molecule can be a fluorescent protein. The fluorescent protein can include but is not limited to Allophycocyanin (APC), B-Phycoerythrin (BPE), R-Phycoerythrin (R-PE), PerCP, and R-Phycocyanin (RPC). In an embodiment, the large molecule can also be an antibody whose specificity differs from that of the rabbit mAb against the SARS-CoV-2 NP, forming a tandem antibody with multiple specificities.

The rabbit mAb against the SARS-CoV-2 NP can have various in vivo and in vitro applications, including but not limited to immunoassays, immunostaining, immunohistochemistry, diagnosis of virus infection associated with SARS-CoV-2, immuno-oncology therapies, and treatment of some infectious diseases caused by SARS-CoV-2. The immunoassays can include enzyme-linked immunosorbent assay (ELISA). The mAb against the SARS-CoV-2 NP can be used in different forms of ELISA. In an embodiment, the rabbit mAb against the SARS-CoV-2 NP disclosed can be used in a direct ELISA. A direct ELISA can be a plate-based immunosorbent assay intended to detect and quantify a specific antigen from or within a complex biological sample. There are varieties of methods for performing direct ELISA. In an embodiment, the antigen, e.g., the nucleocapsid protein of SARS-CoV-2, can be immobilized or adsorbed onto a surface of a plastic plate. In an embodiment, the plastic plate can be a multi-well microtiter plate. In an embodiment, the multi-well microtiter plane can be a 96-well polystyrene plate. In an embodiment, an excessive amount of blocking protein can be added onto the surface to block all the other binding sites. In an embodiment, the blocking protein is bovine serum albumin. In an embodiment, an antibody specific for the antigen, e.g., the nucleocapsid protein of SARS-CoV-2, can be added onto the surface to form a complex with the antigen. In an embodiment, the antibody can be conjugated with an enzyme. In an embodiment, the enzyme can be HRP. After the excess conjugated antibody is washed off, the conjugated antibody bound to the antigen stays. In an embodiment, the conjugated antibody catalyzes a reaction with an added substrate, resulting in a visible colorimetric output that can be measured by a spectrophotometer or absorbance microplate reader. Direct ELISA, when compared to other forms of ELISA testing, can be performed faster because only one antibody is used, and fewer steps are required. In an embodiment, the direct ELISA can test specific antibody-to-antigen reactions and helps to eliminate cross-reactivity between other antibodies. Direct ELISA is suitable for qualitative and quantitative antigen detection in samples of interest, antibody screening, and epitope mapping.

The binding Kinetics 11B12, 5B4, 1F8, 1G7, 3A7, 6H7, and 5B12 are summarized in Table 1. As seen in Table 1, 11B12, 5B4, 1F8, 1G7, 3A7, 6H7, and 5B12 exhibit excellent binding affinity and specificity to the nucleocapsid protein of SARS-CoV-2, as evidenced by their dissociate constants (K_{D}) of 5.65E-11, 7.17E-10, 2.29E-09, 8.14E-09, 1.22E-09, 6.74E-10, 1.89E-09, respectively.

TABLE 1
The binding Kinetics 11B12, 5B4,
1F8, 1G7, 3A7, 6H7, and 5B12

| Clone | K_{off}(l/s) | Kₒₙ(l/Ms) | K_{D}(M) |
|---|---|---|---|
| 1F8 | 2.06E−04 | 9.02E+04 | 2.29E−09 |
| 1G7 | 9.87E−05 | 1.21E+04 | 8.14E−09 |
| 3A7 | 2.72E−04 | 2.24E+05 | 1.22E−09 |
| 5B4 | 2.61E−04 | 3.64E+05 | 7.17E−10 |
| 5B12 | 8.29E−05 | 4.40E+04 | 1.89E−09 |
| 6H7 | 2.38E−04 | 3.54E+05 | 6.74E−10 |
| 11B12 | 2.63E−05 | 4.65E+05 | 5.65E−11 |

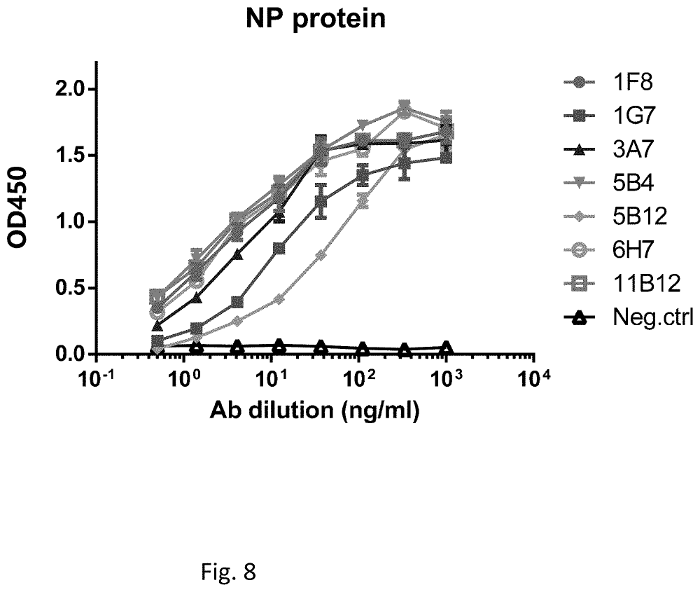

Referring to FIG. 8, FIG. 8 shows curves of 11B12, 5B4, 1F8, 1G7, 3A7, 6H7, and 5B12 in a direct antigen ELISA for detecting the SARS-CoV-2 NP, in accordance with an embodiment. The X-axis shows antibody concentration at a unit of ng/ml, and the Y-axis shows optical density at the wavelength of 450 nm (OD₄₅₀). As seen, all the rabbit mAbs against the SARS-CoV-2 NP exhibit excellent “S” curves. The negative control was conducted under the same procedures as the rabbit mAbs against the SARS-CoV-2, except that a blank buffer is used instead of the rabbit mAbs against the SARS-CoV-2 NP. The blank buffer is the buffer for diluting the rabbit mAbs. Compared to the curve for the negative control, 11B12, 5B4, 1F8, 1G7, 3A7, 6H7, and 5B12 exhibit excellent sensitivity to a wide range of concentrations of nucleocapsid protein of SARS-CoV-2.

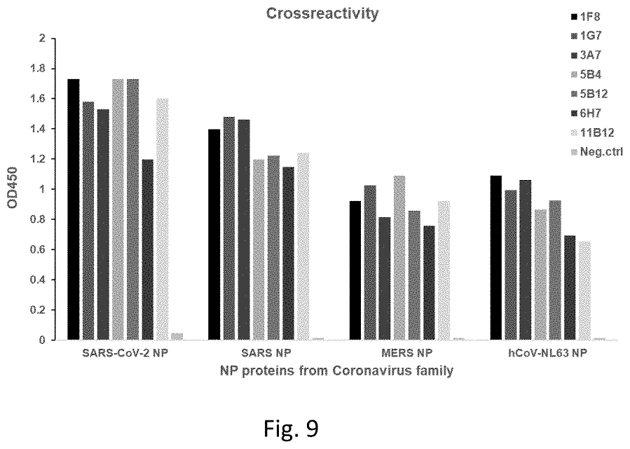

Referring to FIG. 9, FIG. 9 shows cross-reactivity of 11B12, 5B4, 1F8, 1G7, 3A7, 6H7, and 5B12 to nucleocapsid proteins of SARS-CoV-2 (SARS-CoV-2), SARS, MERS, hCoV-NL63, in accordance with an embodiment. Y-axis shows optical density at 450 nm of a direct ELISA assay. Each set of columns includes eight (8) columns that are arranged in an order along the X-axis direction to show the cross-reactivity of 1F8, 1G7, 3A7, 5B4, 5B12, 6H7, 11B12, and negative control, respectively, for the antigen such as SARS-CoV-2 NP, SARS NP, MERS NP, and hCov-NL63 NP. 11B12, 5B4, 1F8, 1G7, 3A7, 6H7, and 5B12 can detect the NP from SARS-CoV-2, SARS, MERS, and hCov-NL63. Each of 11B12, 5B4, 1F8, 1G7, 3A7, 6H7, and 5B12 exhibits the strongest detection signal, i.e., the highest OD450, in detecting SARS-CoV-2 NP, second strongest detection signal in detecting SARS, and weakest detection signal in detecting hCov-NL63.

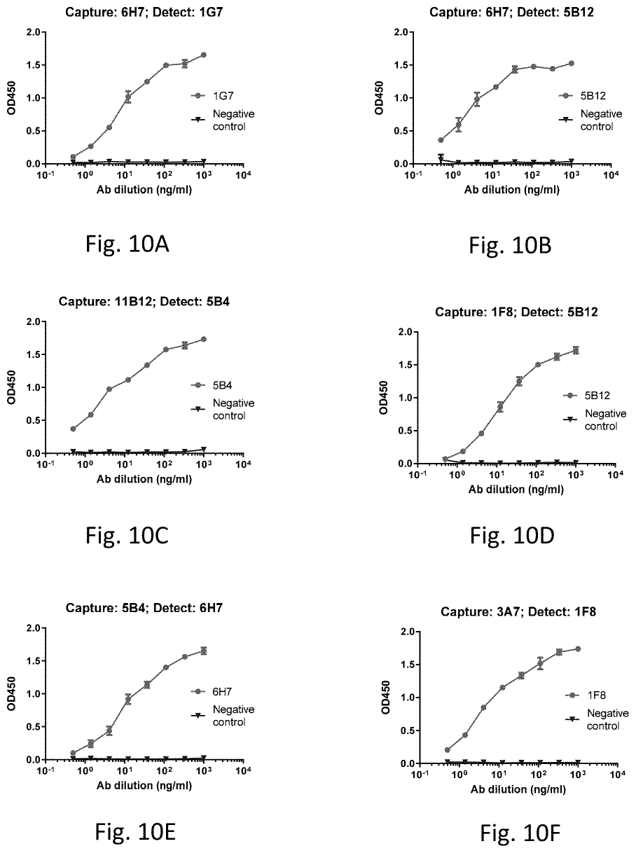

FIG. 10 A shows results of sandwich ELISA using 6H7 as the capture antibody and 1G7 as the detection antibody, in accordance with an embodiment. As seen, 3B8 can bind or detect to NP of SARS-CoV-2 after the NP is bound by 6H7.

FIG. 10 B shows results of sandwich ELISA using 6H7 as the capture antibody and 5B12 as the detection antibody, in accordance with an embodiment. As seen, 5B12 can bind or detect to NP of SARS-CoV-2 after the NP is bound by 6H7.

FIG. 10 C shows results of sandwich ELISA using 11B12 as the capture antibody and 5B4 as the detection antibody, in accordance with an embodiment. As seen, 5B4 can bind or detect NP of SARS-CoV-2 after the NP is bound by 11B12.

FIG. 10 D shows results of sandwich ELISA using 1F8 as the capture antibody and 5B12 as the detection antibody, in accordance with an embodiment. As seen, 5B12 can bind or detect NP of SARS-CoV-2 after the NP is bound by 1F8.

FIG. 10 E shows results of sandwich ELISA using 5B4 as the capture antibody and 6H7 as the detection antibody, in accordance with an embodiment. As seen, 6H7 can bind to NP of SARS-CoV-2 after the NP is bound by 5B4.

FIG. 10 F shows results of sandwich ELISA using 3A7 as the capture antibody and 1F8 as the detection antibody, in accordance with an embodiment. As seen, 1F8 can bind to NP of SARS-CoV-2 after the NP is bound by 3A7.

### Methods

1. Generation, isolation, and purification of rabbit monoclonal antibodies against the SARS-CoV-2 NP.

The rabbit mAbs against the SARS-CoV-2 NP can be generated by various techniques, including monoclonal antibody methodology, e.g., the somatic cell hybridization technique and other techniques including but not limited to viral or oncogenic transformation of B lymphocytes. In an embodiment, the recombinant rabbit mAbs are generated by single B cell-based technology. Briefly, the SARS-CoV-2 NP protein emulsified in incomplete Freund's adjuvant (IFA) was the immunogen to stimulate an immune response in New Zealand White rabbits (4-6 weeks of age) via subcutaneous injection. Pre and post-immunization sera were collected on days 0, 14, 28, 42, and 69, respectively. The spleen was harvested from the rabbit. Fresh single splenocytes were isolated and cultured overnight in a B cell medium, e.g., a B cell medium from Yurogen Biosystems, China. A fresh single-cell suspension of splenocytes in PBS supplemented with 2% FBS and 1 mM EDTA was prepared before single-cell sorting. Splenocytes were processed using the single-B-cell based SMab® platform (Yurogen Biosystems, China) and sorted one cell per well on a FACS Aria II (BD Biosciences, USA) into 96-well plates. S1-specific primary B cells were cultured in a B cell complete medium, e.g., a rabbit B cell complete medium from Yurogen Biosystems, China, for 10-14 days at 37° C. with 5% CO₂. At the end of primary B cell culture, NP-recognizing B cell clones were identified by screening primary B cell culture supernatants against NB by direct ELISA. Positive B-cell clones were determined by OD450 nm values more than 5-fold over background noise. The variable region of IgG heavy chain and light chain from top positive clones was recovered by RT-PCR. The full-length IgG heavy and light chains of each clone were co-transfected into HEK293T cells. The supernatants containing recombinant rabbit IgG from transfected HEK293T were screened for their specificity to the NP by ELISA. The PCR fragments of variable regions of selected clones were cloned into pcDNA3.4 vector for antibody expression in HEK293F cells.

The rabbit monoclonal antibodies can be isolated and purified by a variety of techniques. In an embodiment, rabbit monoclonal antibodies can be isolated from the culture supernatant from mammalian cells transfected with rabbit antibody genes, subsequently purified by Protein A affinity chromatography. The purity and function of purified rabbit monoclonal antibodies can be verified by SDS-PAGE and ELISA, respectively.

2. Preparation of conjugated rabbit monoclonal antibodies against the SARS-CoV-2 NP. Rabbit monoclonal antibodies were biotinylated using Pierce EZ-Link Sulfo-NHS-Biotin in accordance with the manufacturer manual. Briefly, rabbit monoclonal antibodies in 1×PBS, pH 7.4 were incubated with Sulfo-NHS-Biotin for 30 minutes at room temperature.

3. ELISA for characterization of immunized rabbit sera and monoclonal antibodies against the SARS-CoV-2 NP. Antigens such as the SARS-CoV-2 NP or NPs from other viruses were coated on high binding ELISA plates (e.g., Corning, Cat No: 9018) at 4° C. overnight in 1×PBS, pH7.4. Coated plates were washed three times with washing buffer (1×PBS supplemented with 0.5% (V/V) Tween-20 (Sigma, Cat. no: P9416), and blocked with blocking buffer (1×PBS supplemented with 5% (W/V) skim milk). After blocking, plates were incubated with serial diluted rabbit serum samples or monoclonal antibody for 1 hour at room temperature, followed by washing five times with the washing buffer and then incubating with goat anti-rabbit IgG antibody conjugated with HRP, e.g., HRP from Jackson Immuno Research, Cat No: 111-035-045, in the blocking buffer at 1:5000 dilutions. After washing the plates five times with the washing buffer, the plates were developed using a colorimetric reaction catalyzed by HRP. Optical density (OD) values at 450 nm and 630 nm were measured by Epoch microplate spectrophotometer (Biotek, USA). The final value was obtained using OD450 subtracted by OD630. The serum titer was calculated as the maximum dilution where the diluted serum produces OD450 reading of 2-fold or higher than that of the control samples.

4. Capture ELISA with the rabbit mAbs against the SARS-CoV-2 NP. Anti-rabbit IgG Fc antibodies were coated on high binding ELISA plates at 4° C. overnight in 1×PBS, pH7.4. Coated plates were washed with Wash Buffer (1×PBS supplemented with 0.5% (V/V) Tween-20), and blocked with Blocking Buffer (1×PBS supplemented with 5% (W/V) skim milk). After blocking, plates were incubated with the rabbit mAbs against the SARS-CoV-2 NP for 1 hour at room temperature, followed by washing and then incubating with biotinylated SARS-CoV-2 NP serial diluted at three fold. The plates were further incubated with streptavidin conjugated to HRP. After a final wash, plates were developed using a colorimetric reaction catalyzed by HRP to see if a monoclonal antibody can or cannot capture the NP. It is appreciated that the capture ELISA can also be performed with different steps, reagents, experimental parameters from those discussed above.

5. Sandwich ELISA with the rabbit mAbs against the SARS-CoV-2 NP. Capture antibodies were coated on high binding ELISA plates at 4° C. overnight in 0.02M bicarbonate buffer, pH9.4. Coated plates were washed with Wash Buffer (1×PBS supplemented with 0.5% (V/V) Tween-20), and blocked with Blocking Buffer (1×PBS supplemented with 5% (W/V) skim milk). After blocking, plates were incubated with the SARS-CoV-2 NP serial diluted in 3 folds for 1 hour at room temperature, followed by washing and then incubating biotinylated mAbs against the SARS-CoV-2 NP. The plates were further incubated with streptavidin conjugated to HRP. After a final wash, plates were developed using a colorimetric reaction catalyzed by HRP to see if a pair of capture and detection monoclonal antibodies can or cannot bind to the SARS-CoV-2 NP simultaneously. It is appreciated that the sandwich ELISA can also be performed with different steps, reagents, experimental parameters from those discussed above.

6. Determination of the binding kinetics of the rabbit mAbs against the SARS-CoV-2 NP. The binding kinetics of the rabbit mAbs against the SARS-CoV-2 NP were analyzed by surface plasmon resonance (SPR) using a BIAcore instrument with Protein A sensor chip (GE Healthcare, USA). All experiments were performed at 25° C. at a flow rate of 40 μL/min. The running buffer was degassed PBS with 0.005% Tween-20. Channel 1 was loaded with a reference antibody without specific binding to the antigens used, and channels 2, 3, and 4 were loaded with the antibody candidates. Typically, 2 μg/mL of antibody and a quick injection for 20-30 s yielded˜150-250 response units (RU) of antibody coupling with high reproducibility. Antigen was then injected over all the channel surfaces for 5 min for an association phase followed by a 10-minute dissociation phase by buffer rinse. Multiple association/dissociation cycles were performed using antigen dilution series in the range of 1.2-100 nM, as well as a blank buffer. At the end of each cycle, regeneration was performed by a 30-second injection of glycine buffer (pH 2.0, 10 mM), and antibodies were loaded in each channel again. The kinetic curves were double reference subtracted and analyzed to calculate association rate constant, dissociation rate constant, and affinity constants using BIA evaluation 3.2 and 1:1 Langmuir model.

The term “a,” “an,” or “the” cover both the singular and the plural reference unless the context dictates otherwise. The terms “comprising,” “having,” “including,” and “containing” are open-ended terms, which means “including but not limited to,” unless otherwise indicated.

While the disclosure has been illustrated and described in typical embodiments, it is not intended to be limited to the details shown above since various modifications and substitutions can be made without departing from the present disclosure. As such, further modifications and equivalents of the disclosure herein disclosed may occur to persons having ordinary skills in the art using no more than routine experimentation. Such modifications and equivalents of the disclosure herein encompass nuclear acid sequences encoding the amino acid sequences disclosed.

Aspects

Aspect 1. An antibody for recognizing the SARS-CoV-2 NP, comprising:

(a) a V_{H} CDR1 selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and conservative modifications thereof;

(b) a V_{H} CDR2 selected from the group consisting SEQ ID NO: 8, SEQ ID NO: 9, SEQ ID NO: 10, SEQ ID NO: 11, SEQ ID NO: 12, SEQ ID NO: 13, SEQ ID NO: 14, and conservative modifications thereof;

(c) a V_{H} CDR3 selected from the group consisting of SEQ ID NO: 15, SEQ ID NO: 16, SEQ ID NO: 17, SEQ ID NO: 18, SEQ ID NO: 19, SEQ ID NO: 20, SEQ ID NO: 21, and conservative modifications thereof;

(d) a V_{L} CDR1 selected from the group consisting SEQ ID NO: 22, SEQ ID NO: 23, SEQ ID NO: 24, SEQ ID NO: 25, SEQ ID NO: 26, SEQ ID NO: 27, SEQ ID NO: 28, and conservative modifications thereof;

(e) a V_{L} CDR2 selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, and conservative modifications thereof; and

(f) a V_{L} CDR3 selected from the group consisting of SEQ ID NO: 36, SEQ ID NO: 37, SEQ ID NO: 38, SEQ ID NO: 39, SEQ ID NO: 40, SEQ ID NO: 41, SEQ ID NO: 42, and conservative modifications thereof.

Aspect 2. The antibody of aspect 1, wherein

(a) the V_{H} CDR1 includes the amino acid sequence of SEQ ID NO: 1 or a conservative modification thereof;

(b) the V_{H} CDR2 includes the amino acid sequence of SEQ ID NO: 8 or a conservative modification thereof;

(c) the V_{H} CDR3 includes the amino acid sequence of SEQ ID NO: 15 or a conservative modification thereof;

(d) the V_{L} CDR1 includes the amino acid sequence of SEQ ID NO: 22 or a conservative modification thereof;

(e) the V_{L} CDR2 includes the amino acid sequence of SEQ ID NO: 29 or a conservative modification thereof; and

(f) the V_{L} CDR3 includes the amino acid sequence of SEQ ID NO: 36 or a conservative modification thereof.

Aspect 3. The antibody of aspect 1, wherein

(a) the V_{H} CDR1 includes the amino acid sequence of SEQ ID NO: 2 or a conservative modification thereof;

(b) the V_{H} CDR2 includes the amino acid sequence of SEQ ID NO: 9 or a conservative modification thereof;

(c) the V_{H} CDR3 includes the amino acid sequence of SEQ ID NO: 16 or a conservative modification thereof;

(d) the V_{L} CDR1 includes the amino acid sequence of SEQ ID NO: 23 or a conservative modification thereof;

(e) the V_{L} CDR2 includes the amino acid sequence of SEQ ID NO: 30 or a conservative modification thereof; and

(f) the V_{L} CDR3 includes the amino acid sequence of SEQ ID NO: 37 or a conservative modification thereof.

Aspect 4. The antibody of aspect 1, wherein

(a) the V_{H} CDR1 includes the amino acid sequence of SEQ ID NO: 3 or a conservative modification thereof;

(b) the V_{H} CDR2 includes the amino acid sequence of SEQ ID NO: 10 or a conservative modification thereof;

(c) the V_{H} CDR3 includes the amino acid sequence of SEQ ID NO: 17 or a conservative modification thereof;

(d) the V_{L} CDR1 includes the amino acid sequence of SEQ ID NO: 24 or a conservative modification thereof;

(e) the V_{L} CDR2 includes the amino acid sequence of SEQ ID NO: 31 or a conservative modification thereof; and

(f) the V_{L} CDR3 includes the amino acid sequence of SEQ ID NO: 38 or a conservative modification thereof.

Aspect 5. The antibody of aspect 1, wherein

(a) the V_{H} CDR1 includes the amino acid sequence of SEQ ID NO: 4 or a conservative modification thereof;

(b) the V_{H} CDR2 includes the amino acid sequence of SEQ ID NO: 11 or a conservative modification thereof;

(c) the V_{H} CDR3 includes the amino acid sequence of SEQ ID NO: 18 or a conservative modification thereof;

(d) the V_{L} CDR1 includes the amino acid sequence of SEQ ID NO: 25 or a conservative modification thereof;

(e) the V_{L} CDR2 includes the amino acid sequence of SEQ ID NO: 32 or a conservative modification thereof; and

(f) the V_{L} CDR3 includes the amino acid sequence of SEQ ID NO: 39 or a conservative modification thereof.

Aspect 6. The antibody of aspect 1, wherein

(a) the V_{H} CDR1 includes the amino acid sequence of SEQ ID NO: 5 or a conservative modification thereof;

(b) the V_{H} CDR2 includes the amino acid sequence of SEQ ID NO: 12 or a conservative modification thereof;

(c) the V_{H} CDR3 includes the amino acid sequence of SEQ ID NO: 19 or a conservative modification thereof;

(d) the V_{L} CDR1 includes the amino acid sequence of SEQ ID NO: 26 or a conservative modification thereof;

(e) the V_{L} CDR2 includes the amino acid sequence of SEQ ID NO: 33 or a conservative modification thereof; and

(f) the V_{L} CDR3 includes the amino acid sequence of SEQ ID NO: 40 or a conservative modification thereof.

Aspect 7. The antibody of aspect 1, wherein

(a) the V_{H} CDR1 includes the amino acid sequence of SEQ ID NO: 6 or a conservative modification thereof;

(b) the V_{H} CDR2 includes the amino acid sequence of SEQ ID NO: 13 or a conservative modification thereof;

(c) the V_{H} CDR3 includes the amino acid sequence of SEQ ID NO: 20 or a conservative modification thereof;

(d) the V_{L} CDR1 includes the amino acid sequence of SEQ ID NO: 27 or a conservative modification thereof;

(e) the V_{L} CDR2 includes the amino acid sequence of SEQ ID NO: 34 or a conservative modification thereof; and

(f) the V_{L} CDR3 includes the amino acid sequence of SEQ ID NO: 41 or a conservative modification thereof.

Aspect 8. The antibody of aspect 1, wherein

(a) the V_{H} CDR1 includes the amino acid sequence of SEQ ID NO: 7 or a conservative modification thereof;

(b) the V_{H} CDR2 includes the amino acid sequence of SEQ ID NO: 14 or a conservative modification thereof;

(c) the V_{H} CDR3 includes the amino acid sequence of SEQ ID NO: 21 or a conservative modification thereof;

(d) the V_{L} CDR1 includes the amino acid sequence of SEQ ID NO: 28 or a conservative modification thereof;

(e) the V_{L} CDR2 includes the amino acid sequence of SEQ ID NO: 35 or a conservative modification thereof; and

(f) the V_{L} CDR3 includes the amino acid sequence of SEQ ID NO: 42 or a conservative modification thereof.

Aspect 8. An antibody, comprising:

(a) a V_{H} includes an amino acid sequence selected from the group consisting of SEQ ID NO: 43, SEQ ID NO: 44, SEQ ID NO: 45, SEQ ID NO: 46, SEQ ID NO: 47, SEQ ID NO: 48, SEQ ID NO: 49, and conservative modifications thereof; and

(b) a V_{L} includes an amino acid sequence selected from the group consisting of SEQ ID NO: 50, SEQ ID NO: 51, SEQ ID NO: 52, SEQ ID NO: 53, SEQ ID NO: 54, SEQ ID NO: 55, SEQ ID NO: 56, and conservative modifications thereof;

Aspect 9. The antibody of aspect 8, wherein the V_{H} includes an amino acid sequence of SEQ ID NO: 43, and the V_{L} includes an amino acid sequence of SEQ ID NO:50.
Aspect 10. The antibody of aspect 8, wherein the V_{H} includes an amino acid sequence of SEQ ID NO: 44, and the V_{L} includes an amino acid sequence of SEQ ID NO:51.
Aspect 11. The antibody of aspect 8, wherein the V_{H} includes an amino acid sequence of SEQ ID NO: 45, and the V_{L} includes an amino acid sequence of SEQ ID NO:52.
Aspect 12. The antibody of aspect 8, wherein the V_{H} includes an amino acid sequence of SEQ ID NO: 46, and the V_{L} includes an amino acid sequence of SEQ ID NO:53.
Aspect 13. The antibody of aspect 8, wherein the V_{H} includes an amino acid sequence of SEQ ID NO: 47, and V_{L} includes an amino acid sequence of SEQ ID NO:54.
Aspect 14. The antibody of aspect 8, wherein the V_{H} includes an amino acid sequence of SEQ ID NO: 48, and the V_{L} includes an amino acid sequence of SEQ ID NO: 56.
Aspect 15. The antibody of aspect 8, wherein the V_{H} includes an amino acid sequence of SEQ ID NO: 49, and the V_{L} includes an amino acid sequence of SEQ ID NO: 56.
Aspect 16. An antibody, comprising a Fab fragment including a heavy chain and a light chain,

wherein the heavy chain includes an amino acid sequence of SEQ ID NO: 57, and the light chain includes an amino acid sequence of SEQ ID NO: 64, or

the heavy chain includes an amino acid sequence of SEQ ID NO: 58, and the light chain includes an amino acid sequence of SEQ ID NO: 65, or

the heavy chain includes an amino acid sequence of SEQ ID NO: 59, and the light chain includes an amino acid sequence of SEQ ID NO: 66, or

the heavy chain includes an amino acid sequence of SEQ ID NO: 60, and the light chain includes an amino acid sequence of SEQ ID NO: 67, or

the heavy chain includes an amino acid sequence of SEQ ID NO: 61, and the light chain includes an amino acid sequence of SEQ ID NO: 68, or

the heavy chain includes an amino acid sequence of SEQ ID NO: 62, and the light chain includes an amino acid sequence of SEQ ID NO: 69, and

the heavy chain includes an amino acid sequence of SEQ ID NO: 63, and the light chain includes an amino acid sequence of SEQ ID NO: 70.

Aspect 17. The antibody as in any one of aspects 1-16, wherein the antibody further comprises a covalently or non-covalently attached conjugate.
Aspect 18. The antibody of aspect 17, wherein the conjugate includes an enzyme, a fluorophore, biotin, or streptavidin, or a combination thereof.
Aspect 19. The antibody as in any one of aspects 1-18, wherein the antibody is a humanized or chimeric antibody.
Aspect 20. An ELISA kit for detection of SARS-CoV-2 NP, comprising the antibody as in any one of aspects 1-19.
Aspect 21. A method for detecting SARS-CoV-2, comprising:

adding an antibody as in any one of aspects 1-18.

Aspect 22. The method of aspect 21, wherein the method is a direct ELISA.
Aspect 23. The method of aspect 22, wherein the method is a capture ELISA.
Aspect 24. The method of aspect 24, wherein the method is a sandwich ELISA.

We claim:

1. A monoclonal antibody, comprising:
(a) a V_{H} CDR1 having the amino acid sequence of SEQ ID NO: 1, a V_{H} CDR2 having the amino acid sequence of SEQ ID NO: 8, a V_{H} CDR3 having the amino acid sequence of SEQ ID NO: 15, a V_{L} CDR1 having the amino acid sequence of SEQ ID NO: 22, a V_{L} CDR2 having the amino acid sequence of SEQ ID NO: 29, and a V_{L} CDR3 having the amino acid sequence of SEQ ID NO: 36; or
(b) a V_{H} CDR1 having the amino acid sequence of SEQ ID NO: 2, a V_{H} CDR2 having the amino acid sequence of SEQ ID NO: 9, V_{H} CDR3 having the amino acid sequence of SEQ ID NO: 16, a V_{L} CDR1 having the amino acid sequence of SEQ ID NO: 23, a V_{L} CDR2 having the amino acid sequence of SEQ ID NO: 30, and a V_{L} CDR3 having the amino acid sequence of SEQ ID NO: 37; or
(c) a V_{H} CDR1 having the amino acid sequence of SEQ ID NO: 3, a V_{H} CDR2 having the amino acid sequence of SEQ ID NO: 10, V_{H} CDR3 having the amino acid sequence of SEQ ID NO: 17, a V_{L} CDR1 having the amino acid sequence of SEQ ID NO: 24, a V_{L} CDR2 having the amino acid sequence of SEQ ID NO: 31, and a V_{L} CDR3 having the amino acid sequence of SEQ ID NO: 38; or
(d) a V_{H} CDR1 having the amino acid sequence of SEQ ID NO: 4, a V_{H} CDR2 having the amino acid sequence of SEQ ID NO: 11, a V_{H} CDR3 having the amino acid sequence of SEQ ID NO: 18, a V_{L} CDR1 having the amino acid sequence of SEQ ID NO: 25, a V_{L} CDR2 having the amino acid sequence of SEQ ID NO: 32, and a V_{L} CDR3 having the amino acid sequence of SEQ ID NO: 39; or
(e) a V_{H} CDR1 having the amino acid sequence of SEQ ID NO: 5, a V_{H} CDR2 having the amino acid sequence of SEQ ID NO: 12, a V_{H} CDR3 having the amino acid sequence of SEQ ID NO: 19, a V_{L} CDR1 having the amino acid sequence of SEQ ID NO: 26, a V_{L} CDR2 having the amino acid sequence of SEQ ID NO: 33, and a V_{L} CDR3 having the amino acid sequence of SEQ ID NO: 40; or
(f) a V_{H} CDR1 having the amino acid sequence of SEQ ID NO: 6, a V_{H} CDR2 having the amino acid sequence of SEQ ID NO: 13, a V_{H} CDR3 having the amino acid sequence of SEQ ID NO: 20, a V_{L} CDR1 having the amino acid sequence of SEQ ID NO: 27, a V_{L} CDR2 having the amino acid sequence of SEQ ID NO: 34, and a V_{L} CDR3 having the amino acid sequence of SEQ ID NO: 41;
(g) a V_{H} CDR1 having the amino acid sequence of SEQ ID NO: 7, a V_{H} CDR2 having the amino acid sequence of SEQ ID NO: 14, a V_{H} CDR3 having the amino acid sequence of SEQ ID NO: 21, a V_{L} CDR1 having the amino acid sequence of SEQ ID NO: 28, a V_{L} CDR2 having the amino acid sequence of SEQ ID NO: 35, and a V_{L} CDR3 having the amino acid sequence of SEQ ID NO: 42.

2. The monoclonal antibody of claim 1, comprising:
(a) a V_{H} comprising the amino acid sequence of SEQ ID NO: 43 and a V_{L} comprising the amino acid sequence of SEQ ID NO: 50; or
(b) a V_{H} comprising the amino acid sequence of SEQ ID NO: 44 and a V_{L} comprising the amino acid sequence of SEQ ID NO: 51; or
(c) a V_{H} comprising the amino acid sequence of SEQ ID NO: 45 and a V_{L} comprising the amino acid sequence of SEQ ID NO: 52; or
(d) a V_{H} comprising the amino acid sequence of SEQ ID NO: 46 and a V_{L} comprising the amino acid sequence of SEQ ID NO: 53; or
(e) a V_{H} comprising the amino acid sequence of SEQ ID NO: 47 and a V_{L} comprising the amino acid sequence of SEQ ID NO: 54; or
(f) a V_{H} comprising the amino acid sequence of SEQ ID NO: 48 and a V_{L} comprising the amino acid sequence of SEQ ID NO: 55; or
(g) a V_{H} comprising the amino acid sequence of SEQ ID NO: 49 and a V_{L} comprising the amino acid sequence of SEQ ID NO: 56.

3. The monoclonal antibody of claim 1, comprising:
(a) a heavy chain comprising the amino acid sequence of SEQ ID NO: 57 and a light chain comprising the amino acid sequence of SEQ ID NO: 64; or
(b) a heavy chain comprising the amino acid sequence of SEQ ID NO: 58 and a light chain comprising the amino acid sequence of SEQ ID NO: 65; or
(c) a heavy chain comprising the amino acid sequence of SEQ ID NO: 59 and a light chain comprising the amino acid sequence of SEQ ID NO: 66; or
(d) a heavy chain comprising the amino acid sequence of SEQ ID NO: 60 and a light chain comprising the amino acid sequence of SEQ ID NO: 67; or
(e) a heavy chain comprising the amino acid sequence of SEQ ID NO: 61 and a light chain comprising the amino acid sequence of SEQ ID NO: 68; or
(f) a heavy chain comprising the amino acid sequence of SEQ ID NO: 62 and a light chain comprising the amino acid sequence of SEQ ID NO: 69; or
(g) a heavy chain comprising the amino acid sequence of SEQ ID NO: 63 and a light chain comprising the amino acid sequence of SEQ ID NO: 70.

4. The monoclonal antibody of claim 1, wherein the antibody further comprises a covalently or non-covalently attached conjugate.

5. The monoclonal antibody of claim 4, wherein the conjugate includes an enzyme, a fluorescence protein, a fluorophore, a biotin, or a streptavidin.

6. The monoclonal antibody of claim 4, wherein the enzyme includes HRP.

7. The monoclonal antibody of claim 1, wherein the monoclonal antibody is a humanized or chimeric antibody.

8. A kit for detecting SARS-CoV-2 or a nucleocapsid protein of SARS-CoV-2, comprising the monoclonal antibody of claim 1.

9. A method for detecting SARS-CoV-2 or a nucleocapsid protein of SARS-CoV-2, comprising:
adding the antibody of claim 1.

10. The method of claim 9, wherein the method is a direct ELISA, and the antibody detects the nucleocapsid protein of SARS-CoV-2.

11. The method of claim 10, wherein the method is an indirect ELISA, capture ELISA, or sandwich ELISA.
